(19) United States
(12) Patent Application Publication
(10) Pub. No.: US 2006/0051362 A1
(43) Pub. Date: Mar. 9, 2006

(54) Antigens for actinobacillus pleuropneumoniae and methods thereof

(76) Inventors: Mario Jacques, Mont Saint-Hilaire (CA); James W Coulton, Westmount (CA); Leonie G Mikael, Montreal (CA); Ali Khamessan, Montreal (CA); Marc Sirois, Trois-Rivieres (CA)

Correspondence Address: DAVID S. RESNICK, 100 SUMMER STREET, NIXON PEABODY LLP, BOSTON, MA, 02110-2131, US

(21) Appl. No.: 10/499,290

(22) PCT Filed: Dec. 18, 2002
(86) PCT No.: PCT/CA02/01963

§ 371 Date: Mar. 10, 2005

Related U.S. Application Data
(60) Provisional application No. 60/340,483, filed on Dec. 18, 2001.

(51) Int. Cl. G01N 33/554; C07H 21/04; C12P 21/06; A61K 39/02; C07K 14/195

(52) U.S. Cl. 424/190.1; 435/7.32; 435/69.1; 435/471; 435/252.3; 530/350; 536/23.7

(57) ABSTRACT

The present invention is related to a DNA sequence of outer membrane proteins of Actinobacillus pleuropneumoniae expressed in a subject suffering from pleuropneumonia, the sequence coding for the receptor for the uptake of ferric hydroxamate FhuA as set forth in SEQ ID NO: 1 in Actinobacillus pleuropneumoniae, or any functional fragment thereof. The present invention also relates to a DNA sequence of outer membrane proteins of Actinobacillus pleuropneumoniae expressed in a subject suffering from pleuropneumonia, the sequence coding for a hemoglobin-binding protein as set forth in SEQ ID NO: 4 in Actinobacillus pleuropneumoniae, or any functional fragment thereof. The present invention further relates to composition, diagnostic kit and methods using the DNA sequences of the present invention.

### BACKGROUND OF THE INVENTION

(a) Field of the Invention

This invention relates to DNA sequences coding for Actinobacillus pleuropneumoniae antigens, pharmaceutical compositions comprising the antigens encoded by these DNA sequences, the use of this composition as a vaccine and, kit and method for diagnostic of porcine pleuropneumonia infection.

(b) Description of Prior Art

Actinobacillus pleuropneumoniae is the etiologic agent of porcine pleuropneumonia, a highly contagious respiratory disease with major economic implications for the swine industry worldwide. Infection by A. pleuropneumoniae is a multifactorial process governed by many virulence factors acting alone or in concert to establish the pathogen in the porcine host. Iron has long been associated with bacterial virulence either as a requirement for bacterial growth, or acting as an environmental signal that regulates the expression of other virulence factors. The scarce bioavailability of iron (10⁻¹⁸ M) at concentrations lower than are required for most bacterial growth (10⁻⁶ to 10⁻⁸ M) necessitates that bacteria utilize mechanisms for high-affinity iron acquisition.

A. pleuropneumoniae is capable of using hemoglobin, hemin-containing compounds, and porcine transferrin as sources of iron for growth. In addition, this bacterium can produce hemolysins, toxins belonging to the RTX group. All of these factors may contribute to the virulence of the bacterium. A. pleuropneumoniae also responds to iron-restricted conditions by inducing the synthesis of a specific subset of OMPs including two membrane-bound transferrin-specific receptors called TbpA and TbpB (Wilke M. et al. 1997 J. Vet Med. B 44(2):73-86). Although it was tempting to speculate that one of these proteins might serve as a receptor for some siderophore, preliminary bioassays by Deneer et al. (Deneer et al. 1989. Infect. Immun. 57:798-804) did not demonstrate any siderophore production in A. pleuropneumoniae. However, it was suggested that A. pleuropneumoniae might obtain iron in vivo directly from host sources in a manner similar to that of Neisseria species which apparently also do not produce siderophores. When Diarra et al. (Diarra, M. S. et al. 1996. Appl. Environ. Microbiol. 62:853-959) tested the ability of all serotypes of A. pleuropneumoniae to use different exogenous sources of iron (specifically catecholates and hydroxamates), growth promotion assays showed that all strains of A. pleuropneumoniae tested (except one field strain of serotype 5) were capable of using ferrichrome as a growth promoting substance under iron-limited conditions. They also demonstrated that A. pleuropneumoniae strain 87-682 of serotype 1 and strain 2245 of serotype 5 secreted into the culture medium an iron chelator in response to iron stress. However, this potential A. pleuropneumoniae siderophore had a structure that did not conform to the well-characterized assay for catechols for hydroxamates. Noteworthy however is that some bacteria are known to use siderophores that are produced by other microorganisms, and hence these bacteria must have the necessary receptors for the assimilation of different siderophores.

Several fungi including Ustilago sphaerogena synthesize ferrichrome, a hydroxamate siderophore. The ferric hydroxamate uptake (fhu) system in E. coli is well recognized as one of the paradigms for siderophore transport. The E. coli fhu system consists of four genes designated fhuA, fhuC, fhuD and fhuB; they are arranged in one operon at minute 3 of the linkage map and they are transcribed clockwise in this order. The FhuA gene encodes the multifunctional outer membrane protein FhuA (79 kDa) which acts in E. coli as the ferrichrome-iron receptor as well as the receptor for phages T1, T5, φ80 and UC-1, for the bacterial toxin colicin M and for some antibiotics such as albomycin (a structural analog of ferrichrome) and rifamycin CGP 4832. It is a key player in the ferric hydroxamate uptake system, specific for Fe3+-ferrichrome and functioning as a ligand-specific gated channel. Solving the high resolution X-ray crystallographic structure of FhuA from E. coli was a major advance in understanding some of it structure-function relationships (Ferguson, A. D., et al. 1998. Science 282:2215-2220). Periplasmic FhuD (31 kDa), and cytoplasmic-membrane associated FhuC (29 kDa) and FhuB (41 kDa) are proteins necessary for the transport of ferrichrome and other Fe3+-hydroxamate compounds (Fe3+-aerobactin, Fe3+-coprogen) from the periplasm, across the cytoplasmic membrane into the cytoplasm. The protein complex TonB-ExbB-ExbD provides energy for this process.

It is now reported that the genome of A. pleuropneumoniae contains an operon with genes homologous to those of the E. coli ferric hydroxamate uptake system, albeit in a different gene order. The distribution of fhu genes among different serotypes of A. pleuropneumoniae and the expression of the gene coding for the outer membrane protein receptor FhuA were also studied. Structural similarities between FhuA of E. coli and FhuA of A. pleuropneumoniae were deduced by three-dimensional modeling.

Growth of an invading micro-organism within host tissues is an essential feature of the process of infection. This growth depends on a number of factors, including the ability of a given pathogen to acquire various nutrients needed for survival. One such nutrient is iron. Iron is required as a cofactor for many cellular and biochemical processes. Within the host, however, free iron is virtually absent. This is due to its relative insolubility, and to the fact that almost all of the extracellular iron is bound to the iron-binding glycoproteins, lactoferrin and transferrin. Most of the intracellular iron is sequestered by heme-containing proteins, such as hemoglobin (Hb). As a result, iron concentrations within host tissues are too low to support microbial growth. This iron-limitation thus serves as an important first line of defence against bacterial infection. Bacteria, however, make use of a number of strategies in order to carry out the transport of iron into their cytoplasm for use in essential processes.

Gram-negative bacterial cell envelopes are composed of three distinct layers; the outer membrane (OM), the periplasm and the cytoplasmic membrane (CM). The OM consists of an asymmetrical lipid bilayer, with lipopolysaccharide (LPS) as the main component of the external leaflet, and lipids such as phosphatidyl-ethanolamine or phosphatidylglycerol making up the inner leaflet. Within the OM are embedded a number of different classes of proteins. Two examples of these are the non-specific porins which participate in controlling the flux of small hydrophilic molecules between the external and internal environments, and the high affinity receptors which are involved in the transport of nutrients such as iron into the cell. The periplasmic space, found between the OM and CM, contains a number of binding proteins that serve to carry nutrients to their cognate receptors on the external surface of the CM. In addition, a layer of peptidoglycan is found in the periplasmic space, and this serves to confer structural rigidity and shape to the cell. The CM is also composed of a lipid bilayer; however, unlike the OM, the CM is symmetrical and is composed mainly of phospholipids. The main function of the CM is to act as an osmotic barrier for the cell. Survival of bacterial cells, therefore, requires proper functioning of each of these layers, including their contribution to the import of nutrients.

The bacterial requirement for iron is, as previously mentioned, absolute. As such, the inability of bacteria to synthesize heme has served as an impetus for the evolution of various high affinity iron-acquisition systems in order to compensate for the lack of free iron within hosts. One of the strategies used is the synthesis and secretion of iron chelators which bind with high affinity to iron. These are known as siderophores. Having captured iron from the external environment, siderophores are subsequently recognized by high-affinity receptors on the bacterial cell surface, internalized and stripped of their iron. Another iron-acquisition mechanism involves the receptor-mediated binding of host iron-binding proteins, such as lactoferrin, transferrin and heme-containing proteins. As mentioned previously, the OM plays a key role in this type of iron-capture strategy. It is also important to note that many of these iron-acquisition systems are themselves iron-regulated; under conditions of iron deprivation, bacteria upregulate the expression of iron-binding protein receptors and heme-protein binding receptors on the external surface of their OMs.

Due to their abundance within the host, heme-containing proteins such as hemoglobin represent a valuable source of iron for many pathogens. Many bacteria are known to utilize heme as their sole source of iron, and the use of heme-containing proteins as a source of iron is thought to occur via two general classes of mechanisms, involving three types of proteins.

In an elaborate system, certain bacteria synthesize and secrete a heme-binding protein known as a hemophore which, having bound heme in the external environment, is recognized by a receptor on the bacterial cell surface. This hemophore system is analogous to the siderophore system used by many bacteria. The second system, also the most well-characterized, involves the direct recognition of heme-containing compounds, such as Hb, by high-affinity outer membrane receptors. Several bacterial species have been shown to express Hb-binding proteins. Among these are Haemophilus ducreyi, Pasteurella multocida and Neisseria spp. In addition, an OMP of Haemophilus influenzae, HxuA, has been demonstrated to bind hemopexin. Interestingly, HxuA has been shown to function at both the OM level, and as a soluble heme-binding protein.

Each of the previously described mechanisms relies on the presence of a third class of protein which serves to degrade heme-sequestering proteins in order to allow the import of the heme molecule alone. The best-characterized example of such a protein is the lysine-specific cysteine protease Kgp of Porphyromonas gingivalis. This protein is capable of binding and degrading soluble Hb, as well as hemopexin, haptoglogin and transferrin. It is also important to note that much of the heme found within the host is located intracellularly, within erythrocytes. Thus, in order for a pathogen to gain access to heme or heme-proteins, some form of cell damage must be carried out. Certain bacteria secrete hemolysins which destroy erythrocytes, leading to the release of heme which is subsequently sequestered by heme-proteins.

The import of heme into the internal environment of the bacterial cell is an energy dependent process. In most gram-negative organisms, the energy for transport of heme across the OM is provided by TonB in association with ExbB and ExbD proteins. This system utilises the proton motive force contained in the voltage difference across the cytoplasmic membrane for the directed passage of ligands into the periplasm. OM receptors that require the energy transduction provided by TonB are classified as “TonB dependent”, and share regions of amino acid homology called “TonB boxes” which are thought to physically interact with TonB. It has been shown in a number of Gram-negative pathogens that the presence of TonB is absolutely essential for the utilisation of heme as an iron source.

Actinobacillus pleuropneumoniae, a gram-negative encapsulated rod and member of the Pasteurellaceae family, is the causative agent of porcine pleuropneumonia. This species is divided into two main biotypes; members of biotype 1 require nicotinamide adenine dinucleotide (NAD) for growth, whereas the members of biotype 2 do not. Biotype 1 is further subdivided into 12 recognised serotypes. The basis for differentiation between these serotypes is the composition of their capsular polysaccharides. In North America, serotypes 1, 5 and 7 are most commonly found, whereas serotype 2 predominates in Europe. Actinobacillus pleuropneumoniae is known to use RTX toxins (two of which exhibit hemolytic activity) as well as LPS, capsular polysaccharides and a number of OMP's for induction of pathogenesis. Several iron-containing compounds have been shown to have the potential to act as the sole iron source for Actinobacillus pleuropneumoniae; these include porcine transferrin (Wilke M. et al. 1997 J. Vet. Med. B 44(2):73-86), exogenous siderophores (Diarra, M. S. et al. 1996. Appl. Environ. Microbiol. 62:853-959), as well as heme compounds liberated from host cells, such as Hb or hemin (Archambault, M. et al. 1999. FEMS Microbiol. Lett. 173: 17-25). As with a large number of the OMPs involved in iron acquisition, the Hb-binding protein is thought to be iron-regulated, showing upregulation under conditions of iron starvation. The mechanisms by which these heme compounds are used for iron-acquisition, however, remain largely unknown. Hb has been shown to bind to Actinobacillus pleuropneumoniae LPS in vitro, suggesting a possible docking function of LPS in the process of Hb-internalisation (Bélanger, M. et al. 1995. Infect. Immun. 63:656-662).

Strategies used to isolate Hb-binding proteins have relied heavily on the use of immobilized-ligand affinity chromatography (Elkins. C 1995. Infect. Immun. 63:1241-1245). In a commercial preparation of Hb-agarose (Sigma), Hb is covalently bound to an inert matrix (agarose beads). Solubilized OMPs of Actinobacillus pleuropneumoniae were applied to a column containing this matrix, allowing separation of proteins with high and low affinity for the immobilized ligand. Early reports on the Hb-binding capabilities of Actinobacillus pleuropneumoniae suggested that a major protein of 75 kDa and minor proteins of 104 kDa, 47 kDa and 36 kDa, expressed in the OM, each bind Hb with high affinity. Haemophilus ducreyi, a close phylogenetic relative of Actinobacillus pleuropneumoniae, has been shown to express a single Hb-binding protein, with a molecular mass of approximately 100 kDa.

Early attempts to obtain amino acid sequence information on the Hb-binding proteins of Actinobacillus pleuropneumoniae were largely unsuccessful.

### SUMMARY OF THE INVENTION

Given that Actinobacillus pleuropneumoniae, a swine pathogen, utilizes ferrichrome as an iron source, the molecular cloning and sequences for genes involved in the uptake of this hydroxamate siderophore are reported herein. In a single operon, four genes fhuC, fhuD, fhuB and fhuA were identified and they encode proteins homologous to proteins of the ferric hydroxamate uptake systems of several bacteria including E. coli. The fhuA gene encodes the 77 kDa outer membrane protein FhuA, receptor for ferrichrome; FhuD is the 35.6 kDa periplasmic protein responsible for the translocation of ferric hydroxamate from the outer to the inner membrane; FhuC (28.5 kDa) and FhuB (69.4 kDa) are cytoplasmic membrane-associated proteins, components of an ABC transporter which internalize the ferric hydroxamate. Reference strains representing the 12 known serotypes of A. pleuropneumoniae all tested positive for the four fhu genes. When A. pleuropneumoniae FhuA was affinity-tagged with His6 at its amino-terminus and expressed in an E. coli host, the recombinant protein reacted with a monoclonal antibody against E. coli FhuA as well as a polyclonal pig serum raised against an A. pleuropneumoniae infection. It was concluded that fhuA is expressed in vivo by A. pleuropneumoniae. Three-dimensional modeling of the outer membrane protein FhuA was achieved by threading it to the X-ray crystallographic structure of the homologous protein in E. coli. FhuA from A. pleuropneumoniae shares the same overall fold as E. coli; it possesses an N-terminal cork domain followed by a C-terminal β-barrel domain and displays eleven extracellular loops and ten periplasmic turns.

Given that the Hb-binding protein is expressed on the bacterial surface, and that it is an essential protein, it is proposed that this protein of A. pleuropneumoniae also represents a potential vaccine candidate. In order to perform analyses in these areas, there is an absolute requirement for the isolation of milligram amounts of purified protein. In this application, outer membrane proteins (OMPs) extracted from cultures of A. pleuropneumoniae grown under iron-restricted conditions were incubated with a matrix of cross-linked agarose, covalently bound to Hb. This analysis revealed the presence of a novel OMP of molecular mass 105 kDa which displays Hb-binding characteristics. In addition, the presence of a minor Hb-binding protein of molecular mass 75 kDa was confirmed. In gel trypsinization was performed on the 105 kDa species in order to generate peptide fragments to be analyzed by mass spectrometry. This examination provided amino acid sequence information regarding three internal segments of the A. pleuropneumoniae Hb-binding protein. Screening work was done in order to determine the appropriate conditions for the generation of a fast performance liquid chromatography-(FPLC) compatible matrix to allow larger scale purification by immobilized-ligand affinity chromatography. It is now reported the molecular cloning and sequence of the gene coding for the 105 kDa Hb-binding protein of A. pleuropneumoniae.

In accordance with the present invention there is provided a DNA sequence of outer membrane proteins of Actinobacillus pleuropneumoniae expressed in a subject suffering from pleuropneumonia, the sequence coding for the receptor for the uptake of ferric hydroxamate FhuA as set forth in SEQ ID NO: 1 in Actinobacillus pleuropneumoniae, or any functional fragment thereof.

In accordance with the present invention, there is also provided a DNA sequence of outer membrane proteins of Actinobacillus pleuropneumoniae expressed in a subject suffering from pleuropneumonia, the sequence coding for a hemoglobin-binding protein as set forth in SEQ ID NO: 4 (illustrated at FIG. 7) in Actinobacillus pleuropneumoniae, or any functional fragment thereof.

The DNA sequence in accordance with a preferred embodiment of the present invention, wherein the subject is a pig.

The DNA sequence in accordance with a preferred embodiment of the present invention, wherein the sequence is present in all serotypes of A. pleuropneumoniae.

In accordance with the present invention, there is provided a DNA construct comprising an expression vector comprising the sequence of the present invention.

The construct in accordance with a preferred embodiment of the present invention, wherein the expression vector is pLM202 or pRSC06.

In accordance with the present invention, there is provided a host cell stably transformed with the construct of the present invention.

The cell in accordance with a preferred embodiment of the present invention, wherein the host is E. coli.

In accordance with the present invention, there is provided a recombinant protein obtained from the cell of the present invention.

In accordance with the present invention, there is provided a method for obtaining recombinant protein from the host cell of the present invention, comprising the steps of:

purifying proteins produced by the host cell of the present invention; and

resuspending the proteins in a detergent.

The method in accordance with a preferred embodiment of the present invention, wherein purification is performed with Ni-NTA column.

The method in accordance with a preferred embodiment of the present invention, wherein resuspension is performed in a detergent selected from the group consisting of Guanidine hydrochoride, urea and Empigen.

In accordance with the present invention, there is provided a pharmaceutical composition for immunoprotecting a subject against Actinobacillus pleuropneumoniae, which comprises an amount of antigen encoded by the DNA sequence of the present invention sufficient to elicit a protecting immune response from the subject in association with a pharmaceutically acceptable carrier.

The composition in accordance with a preferred embodiment of the present invention, wherein the carrier is for administration by a route selected from the group consisting of parenteral, oral, rectal, intranasal, topical, by instillation into the eye and by inhalation of an aerosol.

In accordance with the present invention, there is provided the use of the pharmaceutical composition of the present invention as a vaccine against porcine pleuropneumonia.

In accordance with the present invention, there is provided an ELISA diagnostic kit for the assay of Actinobacillus pleuropneumoniae antibodies in a biological sample of pigs, comprising, at least one of the following:

a) a plate having bound thereto a purified A. pleuropneumoniae antigen for binding to anti-A. pleuropneumoniae antibodies present in the biological sample of pigs;

b) a positive control vial of biological sample from pigs experimentally inoculated with a strain of A. pleuropneumoniae;

c) a negative control vial of pig biological sample from A. pleuropneumonia free herd; and

d) a detectably labeled conjugate which binds to pig antibodies bound to the plate of a).

The kit in accordance with a preferred embodiment of the present invention, wherein the biological sample is serum.

The kit in accordance with a preferred embodiment of the present invention, wherein the detectably labeled conjugate is an anti-pig antibodies, which is directly or indirectly detected.

The kit in accordance with a preferred embodiment of the present invention, further comprising a substrate which allow the visualization of the detectably labeled conjugate.

The kit in accordance with a preferred embodiment of the present invention, wherein the detectably labeled conjugate comprises an enzyme label.

The kit in accordance with a preferred embodiment of the present invention, wherein the substrate is a composition for providing a colorimetric, fluorimetric or chemiluminescent signal in the presence of the enzyme label.

The kit in accordance with a preferred embodiment of the present invention, wherein the kit is suitable for any Actinobacillus pleuropneumoniae serotypes.

In accordance with the present invention, there is provided a method of diagnosis of A. pleuropneumonia infection in a pig biological sample, which comprises the steps of:

a) subjecting an ELISA plate having bound thereto an antigen encoded by the DNA sequence of the present invention with the pig biological sample; and

b) detecting the presence of A. pleuropneumonia antibodies in said sample; whereby the presence of antibodies is indicative of a A. pleuropneumonia infection.

### BRIEF DESCRIPTION OF THE DRAWINGS

FIGS. 1A-C illustrate the genetic sequence of the complete fhu operon in Actinobacillus pleuropneumoniae serotype 1 comprised of fhuC (bp 892-1656), fhuD (bp 1656-2609), fhuB (2603-4555) and fhuA (4602-6689) (SEQ ID NO:1) and regions flanking the operon.

FIG. 1D illustrates a map of the A. pleuropneumoniae serotype 1 reference strain 4074 gene cluster with homology to fhuACDB of E. coli.;

FIG. 2 illustrates PCR analysis of the fhu operon in A. pleuropneumoniae serotype reference strains using primers (5T3W1 and 5T7W2) flanking the fhu operon and chromosomal A. pleuropneumoniae DNA for template;

FIG. 3 illustrates SDS-polyacrylamide gel electrophoresis (A) and Western Blots (B, C and D) of E. coli BL21 cells expressing recombinant FhuA from A. pleuropneumonia;

FIG. 4 illustrates the structure-based alignment of the A. pleuropneumonia FhuA sequence (SEQ ID NO:2) relative to that of its template for homology modeling, E. coli FhuA (SEQ ID NO:3);

FIG. 5A illustrates a proposed secondary structure of the barrel domain for FhuA of A. pleuropneumoniae;

FIG. 5B illustrates a proposed tertiary structure (C_trace, stereo view) for FhuA of A. pleuropneumonia;

FIG. 6 illustrates SDS-PAGE of outer membrane proteins of Actinobacillus pleuropneumoniae serotype 1 grown under iron-replete and iron-deplete conditions and purified with hemoglobin-agarose;

FIG. 7 illustrates complete genetic sequence of the 2886-bp coding sequence (SEQ ID NO: 4) of the hgbA of Actinobacillus pleuropneumoniae serotype 1;

FIG. 8 illustrates IgG levels against HgbA antigen over time;

FIG. 9 illustrates IgG levels against FhuA antigen over time;

FIG. 10 illustrates IgA levels against FhuA for several dilutions at day 28;

FIG. 11 illustrates IgA levels against HgbA for several dilutions at day 28;

FIG. 12 illustrates Anti-HgbA levels in IgA/pigs at day 28 and with a dilution of 1/100 and

FIG. 13 illustrates Anti-FhuA levels in IgA/pigs at day 28 and with a dilution of 1/100.

### DETAILED DESCRIPTION OF THE INVENTION

In accordance with the present invention, there is provided antigens for Actinobacillus pleuropneumoniae, compositions thereof and diagnostic kit and method for detection and control of porcine pleuropneumonia.

### MATERIALS AND METHODS

Bacterial Strains, Plasmids and Growth Conditions

Strains of E. coli and A. pleuropneumoniae and the plasmids used in this study are listed in Table 1. A. pleuropneumoniae reference strains were grown on brain heart infusion (BHI) (Difco Laboratories, Detroit, Mich.) agar plates supplemented with 15 μg/ml NAD+. E. coli BL21(DE3), E. coli CC118 and E. coli DH5α were grown on Luria-Bertani (LB) Miller media while E. coli strain XL1-Blue MRF′ was grown in NZYCM (GIBCO BRL, Burlington, ON) supplemented with 12.5 μg/ml tetracycline. The phage excision strain of E. coli XLOLR was grown in LB supplemented with 12.5 μg/ml tetracycline. Growth of the phagemid pBK-CMV was carried out for selection on LB media supplemented with 50 μg/ml kanamycin. Strains harboring derivatives of the pET30a+ expression vector were grown on LB media supplemented with 30 μg/ml kanamycin whereas those harboring pGEM derivatives required 50 μg/ml ampicillin. Blue-white colony selection was achieved by the addition of 40 μg/ml 5-bromo-4-chloro-3-indolyl-β-D-galactoside (X-Gal) and 1 mM isopropylthio-β-D-galactoside (IPTG). In order to achieve iron deplete conditions, the culture media were supplemented with 150 μM deferrated ethylenediamine di-hydroxyphenyl acetic acid (EDDHA) (Sigma, Oakville, ON) depleted iron in growth media. Ferrichrome (Sigma) as exogenous source of iron was either spotted onto deferrated media or incorporated into top agar.

TABLE 1
Bacterial strains and plasmids

| Strain | Description | Reference |
|---|---|---|
| E. coli | pho A recA araD139 del(ara-leu)7697 | |
| CC118 | galE galK thi rpsE rpoB argE(am) | |
| E. coli XL1- | Δ(mcrA)183 Δ(mcrCB-hsdSMR-mrr)173 | Stratagene |
| BlueMRF′ | endA1 supE44 thi-1 recA1 gyrA96 | |
| | relA1lac{F′proAB lacl^{q}ZΔM15 | |
| | Tn10(tet^{r})} | |
| E. coli | Δ(mcrA)183 Δ(mcrCB-hsdSMR-mrr)173 | Stratagene |
| XLOLR | endA1 thi−1 recA1 gyrA96 relA1 | |
| | lac{F′proAB lacl^{q}ZΔM15 Tn10(Tet^{r})} | |
| | Su⁻(nonsuppressing) λ^{r} (lambda resistant) | |
| E. coli XL1- | supE44 hsdR17 recA1 endA1 gyrA46 | Stratagene |
| Blue | thi relA1lac⁻F′{proAB⁺lacl^{q} lacZΔM15 | |
| | Tn10(tet^{r}) | |
| E. coli | supE44ΔlacU169(Φ80lacZΔM15)hsdR17 | Stratagene |
| DH5α | recA1 endA1 gyrA96 thi-1 relA1 | |
| E. coli | F⁻ompT hsdS_{B}(r_{B}⁻m_{B}⁻)gal dcm(DE3) | Novagen |
| BL21(DE3) | | |

| | |
|---|---|
| A. pleuropneumoniae serotype1 strain 4074 | Laboratory stock |
| A. pleuropneumoniae serotype 2 strain 4226 | Laboratory stock |
| A. pleuropneumoniae serotype 3 strain 1421 | Laboratory stock |
| A. pleuropneumoniae serotype 4 strain 1462 | Laboratory stock |
| A. pleuropneumoniae serotype 5 strain K-17 | Laboratory stock |
| A. pleuropneumoniae serotype 6 strain FEMO | Laboratory stock |
| A. pleuropneumoniae serotype 7 strain WF83 | Laboratory stock |
| A. pleuropneumoniae serotype 8 strain 405 | Laboratory stock |
| A. pleuropneumoniae serotype 9 strain 13261 | Laboratory stock |
| A. pleuropneumoniae serotype 10 strain 13261 | Laboratory stock |
| A. pleuropneumoniae serotype 11 strain 56153 | Laboratory stock |
| A. pleuropneumoniae serotype 12 strain 8329/85 | Laboratory stock |

| Strain | Description | Reference |
|---|---|---|
| pRSC06 | App 2.8-kb hgbA sequence in pET24 | This |
| | | application |
| pBK-CMV | Km^{r} phagemid, cloning vector of pBR322 | Stratagene |
| | orignin, T3 and T7 promoter | |
| pLM101 | Km^{r}; App 7974-bp fhuCDBA operon in | This |
| | pBK-CMV vector | application |
| pLM201 | Amp^{r}; PCR product of Afor and Arev | This |
| | (mature fhuA) in pGEM | application |
| pET24 | Km^{r} expression vector with C-terminus | Novagen |
| | His-Tag | |
| pET30a+ | Kmr expression vector with N-terminal | Novagen |
| | His-Tag | |
| pLM202 | Kmr; Notl fragment of pLM201 in pET30a | This |
| | | application |
| pHX405 | Ampr, Tetr, pBR322-based plasmid | J. W. |
| | encoding FhuA.H6 of E. coli | Coulton |

Preparation and Solubilization of Outer Membrane Proteins

Outer membrane proteins from A. p. strain 4074 grown under iron-replete or iron-restricted conditions were extracted and solubilized according to the method described by Elkins (Elkins. C 1995. Infect Immun. 63:1241-1245), with some modifications. Cells were harvested by centrifugation at 5000 rpm for 20 min. at 4° C. and re-suspended in phosphate-buffered saline (PBS). These cells were lysed mechanically with the use of a French pressure cell press (Aminco), at an internal pressure of 16 000×g. Intact cells and cell debris were removed by centrifugation at 12 500 rpm for 10 min. at 4° C. The supernatant was then subjected to centrifugation at 100 000×g for 1 hr at 4° C. producing a pellet composed of A. p. total membrane. This pellet was resuspended in 50 mM Tris-Cl pH 7.5, 150 mM NaCl, 1% Sarkosyl and incubated for 1 hour at room temperature (RT) with rocking. The Sarkosyl-insoluble fraction containing outer membrane vesicles was pelleted by centrifugation at 100 000×g, for 1 hr. at 4° C., and the soluble fraction containing cytoplasmic membrane was discarded. This pellet was then resuspended in 50 mM Tris-Cl pH 7.5, 150 mM NaCl, 5 mM EDTA pH 7.5, 1% Zwittergent 3.14 (Buffer 2) and incubated for 1 hr. at 37° C. with rocking. Centrifugation of this suspension at 12 500 rpm for 10 min. at 4° C. yielded at supernatant containing solubilized outer membrane proteins of A. p. This supernatant was preserved at −20° C. for further experimentation.

Affinity Purification with Hb-Agarose

Solubilized OMPs of A. p. were incubated with solid phase bovine Hb-agarose (Sigma) for 1 hr at RT with rocking. The binding protein/Hb-agarose complex was washed with Buffer 2 (described above) in order to remove non-specifically bound proteins. The complex was then washed with a high-salt buffer. (50 mM Tris-Cl pH 7.5, 1 M NaCl, 1% Zw-3.14, 5 mM EDTA pH 7.5) in order to disrupt higher-affinity ionic interactions between Hb and binding-proteins present. Certain experiments required a further wash with either 1, 2, 3 or 4 M guanidine-HCl. At the end of all washing steps, the complex was resuspended in Laemmli sample buffer and boiled for 10 min. in order to carry out sodium-dodecyl-sulfate polyacrylamide gel electrophoresis (SDS-PAGE).

Electrophoresis

SDS-PAGE was conducted according to the method described by Laemmli, with an 8% polyacrylamide running gel. Samples generated during all washing steps were precipitated in 3:1 volumes of acetone, at −80° C. for 2 hrs. and resuspended in small volumes of deionized water. All samples were boiled for 10 min. in solubilization buffer. Broad Range molecular mass markers were obtained from Bio-Rad, and gels were run in a Mini-Protean II vertical slab electrophoresis cell (Bio-Rad) and stained with Coomassie brilliant blue.

Amino Acid Sequence Determination

The major band following affinity purification with bovine Hb-agarose was a 105 kDa OMP species. This band was excised from the SDS-PAGE gel, and in-gel trypsinized using the modified Mann protocol. The trypsinized protein was reconstituted in an appropriate buffer and was subjected to analysis by a QSTAR mass spectrometer equipped with a nanospray source.

Construction of an A. pleuropneumoniae DNA Signal Sequence Library

To screen for exported proteins in A. pleuropneumoniae chromosomal DNA from A. pleuropneumoniae serotype 1 reference strain 4074 was restricted with Sau3AI and separated by agarose gel electrophoresis. DNA fragments corresponding to 500- to 1500-bp were excised and extracted by means of QIAquick Gel Extraction Kit (QIAGEN Inc, Mississauga, ON). The vector containing the truncated ′phoA (pHRM104) was restricted with BamHI, dephosphorylated with shrimp alkaline phosphatase and ligated with A. pleuropneumoniae Sau3AI fragments. Cells from E. coli phoA mutant strain CC118 were transformed with the ligation mixture by electroporation and incubated in LB Miller broth for 1 h. The cells were then plated on LB agar plates containing 500 μg/ml of erythromycin and 50 μg/ml of 5-bromo-4-chloro-3-indolylphosphate (XP). Translocation of alkaline phosphatase across the bacterial inner membrane results in the hydrolysis of XP and the development of blue colony phenotype. This would indicate that fusion proteins were derived from plasmids containing an A. pleuropneumoniae DNA sequence harboring a promoter, a translational start site and a functional signal sequence. Restriction and modification enzymes were purchased from Amersham Pharmacia Biotech (Baie d'Urfé, QC) and Roche Diagnostics (Laval, QC) and used according to the manufacturers' instructions.

Genetic Techniques

Plasmids from the PhoA+ colonies were isolated using the Plasmid QIAprep spin miniprep kit (QIAGEN Inc, Mississauga, ON). The A. pleuropneumoniae DNA insert was sequenced with the oligonucleotide primer foA (Table 2) which hybridizes to the negative strand of phoA. Subsequent primers (F7 and R7; Table 2) were designed from within the plasmid sequence of the positive clone of relevance to this study. An oligonucleotide primer (FA1; Table 2) based on a region of promoter sequences for fhuA from E. coli was also designed. PCR was carried out with standard conditions and varying annealing temperatures, depending on the sequence of the primers used. When the expected PCR product was larger than 5.0 kb, the Expand Long Template PCR System 1 (Roche Diagnostics, Laval, QC) was used rather than Taq polymerase. DNA sequencing of the PCR product was performed at the DNA sequencing core facility at University of Maine (Orono, Me.) by using an ABI model 373A stretch DNA sequencer. Single-stranded synthetic oligonucleotides (Table 2) were synthesized at BioCorp Inc., Montreal, QC. Various PCR products were then digoxigenin (DIG)-labeled using the DIG DNA Labeling and Detection Kit (Roche Diagnostics, Laval, QC) and used as DNA hybridization probes in Southern blots and plaque lift assays. Chromosomal DNA was extracted by the method of Pitcher et al. For Southern blotting experiments, genomic DNA of A. pleuropneumoniae serotypes was restricted with different enzymes, run on 0.7% agarose gel and transferred to positively charged nylon membranes. Conditions of high stringency were applied. Hybridization of the DIG-labeled DNA probes was detected by using phosphatase-labeled anti-DIG antibodies with NBT-BCIP as substrates.

TABLE 2
Sequences of primers used

| | |
|---|---|
| Primer | Sequence |

| | | |
|---|---|---|
| foA | 5′ CACCCGTTAAACGGCGAGCAC 3′ | (SEQ ID NO:5) |
| F7 | 5′ GCAACCGTCCAATCCA 3′ | (SEQ ID NO:6) |
| R7 | 5′ CATCCTGAAACCAAACGA 3′ | (SEQ ID NO:7) |
| FA1 | 5′ CTCGAGCTCGAGGCAGCAGCAGCCGTC | (SEQ ID NO:8) |
| | AGGC 3′ | |
| 5T3W1 | 5′ CATTGAAAACCTCGAGTCG 3′ | (SEQ ID NO:9) |
| 5T7W1 | 5′ GATACCTATCAAGAAGGC 3′ | (SEQ ID NO:10) |
| 5T3W2 | 5′ TGGCGCAACAAAGCAAAT 3′ | (SEQ ID NO:11) |
| 5T7W2 | 5′ GTTGCCTACGTTATCCAC 3′ | (SEQ ID NO:12) |
| 5T3W3 | 5′ ACCGTTGTTCAGCCTTAT 3′ | (SEQ ID NO:13) |
| 5T7W3 | 5′ GAGCGTCCACTTTGTATG 3′ | (SEQ ID NO:14) |
| 5T3W4 | 5′ AGTTGTTGAGTTTGGCGAT 3′ | (SEQ ID NO:15) |
| 5T7W4 | 5′ TCATTGAGTCTGCGCCTT 3′ | (SEQ ID NO:16) |
| 5T3W5 | 5′ TGGTAGCAATAGCGGTCG 3′ | (SEQ ID NO:17) |
| AFor | 5′ GATGAGGTGTCGGTGGTT 3′ | (SEQ ID NO:18) |
| ARev | 5′ TGTGGCATTGACTTTACG 3′ | (SEQ ID NO:19) |
| BFor | 5′ CTTATTCAGCGGATTAGCC 3′ | (SEQ ID NO:20) |
| BRev | 5′ CAACTAATGTGGCGACTAAG 3′ | (SEQ ID NO:21) |
| CFor | 5′ GCAATTCGAGCAGGGTAAG 3′ | (SEQ ID NO:22) |
| CRev | 5′ CCGGTCGTTTGGTTTCAGG 3′ | (SEQ ID NO:23) |
| DFor | 5′ GTGAGCTTCCGTATTTCT 3′ | (SEQ ID NO: 24) |
| DRev | 5′ CGCCTCCGTGCAATAATC 3′ | (SEQ ID NO: 25) |
| Sall/ | GAGGTCGACAACAGTGCATTGGCTCAAGA | (SEQ ID NO:26) |
| HgbA | GC | |
| Xhol/ | GCGCTCGAGGAAAGTAACCTCTGCGGTTA | (SEQ ID NO:27) |
| HgbA | AC | |

Construction and Screening of an A. pleuropneumoniae Phage Bank

A DNA library of A. pleuropneumoniae serotype 1 reference strain 4074 was constructed in lambda Zap Express phage vector (Stratagene, La Jolla, Calif.) following the manufacturer's instructions. Briefly, A. pleuropneumoniae genomic DNA was partially digested with Sau3AI and the resulting DNA fragments were separated by agarose gel electrophoresis. The DNA fragments corresponding approximately from 7 to 12 kb in length were ligated with BamHI-digested lambda Zap Express arms and packaged in vitro with Stratagene Gold II packaging extracts. The E. coli strains used were XL1-Blue MRF′ as the host and XLOLR as the excision plating strain. The library was screened with probes designed for Southern blotting which were DIG-labeled and the positive plaques were again detected with phosphatase-labeled anti-DIG antibodies with NBT-BCIP as substrates. The strongly reacting plaques were purified by successive rounds of screening. After three rounds of screening, the pBK-CMV phagemid of the positive clones was excised using ExAssist Helper Phage (Stratagene). Finally, the plasmids were digested with restriction enzymes (Xbal and Sacl) known to cut once within the multiple cloning site of the vector, thereby evaluating the size(s) of their insert(s). The dideoxynucleotide sequencing reactions were carried out with universal primers T3 and T7. To complete walking the entire sequence of the plasmid insert(s), internal primers were designed (5T3W1, 5T7W1, 5T3W2, 5T7W2, 5T3W3, 5T7W3, 5T3W4, 5T7W4, 5T3W5; Table 2) based on the sequences obtained. Analysis of sequence homology and protein localization. DNA sequence analysis was carried out with the aid of programs from University of Wisconsin Genetics Computer Group Software using the National Center for Biotechnology Information Database. Multiple sequence alignment of proteins employed the Clustal alignment algorithm provided by the Baylor College of Medicine Search Launcher (http://dot.imgen.bcm.tmc.edu:9331/multi-align/multi-align.html). Prediction of protein localization and cleavage sites for signal sequences was carried out by applying the method of Nakai and Kanehisa.

Expression of Recombinant His-Tagged FhuA

The coding sequences (amino acids 1 to 668) of the mature FhuA were PCT-amplified using primers AFor and ARev (Table 2) which were designed to maintain the reading frame of the fhuA gene. The PCR product was then purified with the PCR Purification Kit (QIAGEN), ligated into pGEM®-T Easy Vector (Promega, Madison, Wis.) and transformed into E. coli XL1-Blue cells. The recombinant plasmid pLM201 was treated with NotI and the NotI fragment was cloned directionally and in-frame into the NotI site of pET30a+ expression vector (Novagen, Madison, Wis.) to append a hexahistidine tag at the amino terminus of FhuA. Plasmid pLM202 carrying the mature fhuA sequence tagged with a His6 at its amino terminus was transformed into E. coli DH5α, extracted and purified, and its orientation and reading frame were verified by DNA sequencing. Plasmid pLM202 was then transformed into E. coli BL21 (DE3) (Novagen), the recommended host background for expression of recombinant proteins in pET vectors. Following the manufacturer's suggested procedure, cells containing the recombinant plasmid were grown in broth culture containing kanamycin and induced with IPTG (0.4, 1.0 and 1.5 and 3.0 mM) for the expression of the His-tagged fusion protein. Various times (2, 4, 6, 8, and 24 h) and temperatures (37° C. and 25° C.) were tested for optimal expression of the fusion protein. Whole-cell protein samples were separated by SDS-PAGE following standard procedures and the gels were either stained by Coomassie-Blue or transferred to a nitrocellulose membrane for immunoblotting.

Expression of Recombinant His-Tagged HgbA

To obtain mature HgbA recombinant protein with a His-tag at its carboxy terminus, the following cloning steps were carried out. A 2.8-kb HgbA sequence was amplified using primers Sa1I/HgBA and Xhol/HgbA (Table 2) and the product was cloned into pGEM cloning vector. Next, the plasmid was digested with Sa1I and Xhol enzymes and the resulting SA1I/Xhol fragment and purification of His-HgbA was carried out in the same manner as described for His-FhuA (see above).

Western Blotting

After transfer, membranes were blocked for one hour with a solution of 1% BSA and then incubated overnight at 4° C. with either mouse anti-His6 monoclonal antibody (Roche), or mouse monoclonal antibody against E. coli FhuA (mAb6.1), or immune sera from a pig convalescing from an A. pleuropneumoniae serotype 1 experimental infection. The secondary antibodies used were either a goat anti-mouse IgG+IgM (H+L) or an anti-swine IgG horseradish peroxidase conjugate (Jackson ImmunoResearch Laboratories, West Grove, Pa.). Reactions were revealed by the addition of 4-chloro-1-naphthol and hydrogen peroxide (Sigma).

Homology Model for FhuA of A. pleuropneumoniae

From the gene encoding A. pleuropneumoniae FhuA, the predicted amino acid sequence (residues 1 to 673 of the mature protein) was submitted to the JIGSAW 3D Protein Homology Modelling Server (http://www.bmm.icnet.uk/servers/3djigsaw). The JIGSAW server returned the E. coli FhuA structure, PDB code 1qfg, as the only successful structural template within its sample space. Visual inspection of this homology model revealed a number of discontinuities within extracellular and periplasmic loop regions, Cα pairs in which the distances were too great to form a covalent bond. To better model the loops of A. pleuropneumoniae FhuA, anchor regions flanking these discontinuities were submitted to the CODA server (http://www-cryst.bioc.cam.ac.uk/coda/coda.html). CODA models amino acid sequences of proposed loops by searching against known structures of loop regions within other proteins. The CODA server was able to model all loop regions within the A. pleuropneumoniae FhuA sequence that the JIGSAW server could not successfully model. The acceptable r.m.s. deviation was less than 1.00 Å. The loops and anchor regions modeled by the CODA server are as follows: 158-166, 183-191, 228-236, 291-299, 301-308, 405-412, 538-546, 564-572, 597-604, 610-617, and 630-637.

Nucleotide Sequence Accession Numbers

The entire sequence of the 7974-bp fragment of pLM101 containing the fhuCDBA operon of A. pleuropneumoniae serotype 1 reference strain 4074 described in this study has been deposited in GenBank under the accession number AF351135. The 2886-bp fragment containing hgbA has been deposited under accession number AF468020.

Protein Purification

The expression vectors used were pET30 for FhuA and pET24 for HgbA antigens (NOVAGEN). Plasmids pLM202 (FhuA) and pRSC06 (HgbA) were transformed into E. coli BL21 host cells (NOVAGEN) and the resulting cultures were treated for isolation of inclusion bodies and then their respective recombinant proteins were purified with Ni-NTA Agarose affinity columns (QIAGEN). The proteins were quantified with micro bicinchoninic (BCA) Protein Assay Reagent Kit (PIERCE).

Complexing Process

FhuA and HgbA were processed for intramuscular administration. To that effect, they were incorporated with a commercial oil-based adjuvant (EMULSIGEN, MVP Laboratories Inc, Ralston, Nebr.). The latter is a stable, oil-in-water emulsion designed to be mixed directly with antigens, without the need for further processing.

Immunization Schedule

Materials:

Antigen 1 (FhuA siderophore receptor)
Antigen 2 (HgbA hemoglobin-binding protein)
Eighteen 6-week old SPF piglets
Group I 6 pigs non-vaccinated (NAÏVE)
Group II 6 pigs vaccinated Intramuscular: Antigen 1 (25 μg per dose) complexed with Emulsigen (FE)
Group III 6 pigs vaccinated Intramuscular: Antigen 2 (25 μg per dose) complexed with Emulsigen (HE)

Pigs were randomly assigned to the experimental groups of six pigs each. They were immunized at the beginning of the experiment (Day 0) and two weeks later (Day 14) with 25 μg of the intramuscular preparation (FE, HE) in the neck muscles of the subjects.

Blood samples were obtained for measurement of antibodies by venipuncture of the jugular vein at the time the experiments began (Day 0) as well as 2 weeks (Day 14; prior to the second immunization) and 4 weeks (Day 28; two weeks after the second immunization) after.

Immunological Methods

Sera were isolated from the blood samples collected by conventional methods. All serum samples were titrated in ELISA to quantitate serum IgG and IgA against each of the protein antigens. The ELISA plates used were 96-well Nunc-Immuno Plate Maxisorp (InterMed).

Separate plates for each antigen were coated with 1 μg recombinant protein per well diluted in carbonate buffer pH 9.6 and incubated overnight at 4° C. The following morning, the plates were washed with PBS-Tween20 (0.05%) and then blocked with 0.5% gelatin for 1 hour followed by another round of 3 washes. Sera were diluted 1/40, 1/160, 1/640 and 1/2560 for IgG and 1/40, 1/100, 1/200, 1/400 for IgA detection, in a total volume of 200 μl per well.

For detection of serum IgG, the conjugate used was peroxidase-conjugated goat anti-swine IgG. To detect serum IgA, a mouse anti-porcine IgA was used as a secondary antibody, followed by a biotin goat anti-mouse IgG, an Avidin-peroxidase conjugate. All intermittent incubation steps were followed by three washes with PBS-Tween20. Detection was achieved for all plates by the addition of 2,2′-Azino-bis-3-ethylbenz-thiazoline-6-sulfonic acid (ABTS) with H202 and the plates were allowed to develop for up to 60 min before reading the OD at a wavelentgh of 410 nm.

### RESULTS

Isolation of a Hb-Binding Protein.

Batch-binding affinity purification experiments revealed the presence of an A. p. OMP of molecular mass 105 kDa possessing Hb-binding properties. In addition, a smaller protein with a molecular mass of approximately 75 kDa was visualized on a SDS-PAGE gel stained with Coomassie brilliant blue following affinity purification; expression of the 105 kDa protein was more than tenfold greater than that of the 75 kDa species. Subsequent analysis revealed that the addition of 150 μM deferrated EDDA and 5 μg*ml⁻¹ hemin to the medium induced an increase in expression of several of the A. p. OMPs, including the 105 kDa species.

Determination of Appropriate Conditions for the Elution of Hb-Binding Protein from a Solid Matrix

Multiple washing steps were utilized in order to elute the protein of interest from the affinity matrix. Initial washing steps using the protein solubilization buffer (Buffer 2, described above) removed a large number of non-specifically bound proteins. Washing with a high-salt (1 M NaCl) buffer was ineffective in disrupting the interactions between Hb and the Hb-binding proteins. Elution with a chaotropic agent (2 M guanidine HCl) was able to induce the partial removal of the protein of interest from the matrix, as visualized on a SDS-PAGE gel, stained by the silver-staining technique. Increased in guanidine-HCl concentration, up to 4 M, showed no increase in elution.

Peptide Sequencing and Cloning of hgbA

The 105-kDa outer membrane protein obtained by affinity chromatography was run on SDS-PAGE, transferred to a PVDF membrane for N-terminal amino acid sequencing and the following peptide sequence was obtained QEQMQLDTVKV (SEQ ID NO:28). Tryptic in-gel digestion of the 105-kDa protein followed by Mass Spectroscopy allowed the identification of internal amino acid sequences for 3 peptides (A) NSVEITLK (SEQ ID NO:29) (B) APTSDELYFTFK (SEQ ID NO:30) and (C) FGVDVYVTR (SEQ ID NO:31) (FIG. 6). These peptide segments from App HgbA showed identity with sequences in known TonB-dependent hemoglobin receptors. The positioning of these internal peptides were determined with respect to the position of their homologous peptides within the same gene in P. multocida. Based on the peptide sequences obtained from the amino terminus and internal segments, oligonucleotide primers were designed which allowed the amplification of a 2.8 kb PCR product in App serotype 1. This stretch of nucleotides encoded an incomplete reading frame of 785 amino acids showing identity with hemoglobin binding proteins (HgbA) of several bacteria including P. multocida. Given that sequence information on the carboxy terminus of the hgbA homologue in App was still missing, an oligonucleotide probe was designed from the 3′-end of the 2.4-kb sequence information obtained. This probe was used to screen a genomic library of App and after three rounds of purification, the plasmid of the positive clone was excised and its insert sequenced. The complete sequence for the hgbA of App was thus obtained; it stretches over 2886 bp and encodes an open reading frame of 962 amino acids.

Alignment of hgbA of App and hemoglobin binding proteins from P. multocida, H. influenzae, H. ducrey and A. actinomycetemcomitans revealed high homology in both amino and carboxy termini of the protein from all five bacteria. A putative TonB-box was also assigned for hbgA of App composed of the peptide sequence LDTVIV. Analysis of sequences upstream of hgbA revealed a potential Fur-box as well as an RBS and −35 and −10 sequences in the promoter region. Two sets of primers that amplify 350- and 750-bp regions within hgbA of serotype 1 were tested in PCR and Southern Hybridization experiments with DNA representing the 12 known serotypes of App and all showed a positive signal.

Cloning of the Fhu Operon in A. pleuropneumoniae

By using a truncated gene for alkaline phosphatase (′phoA) that lacks a functional signal sequence, a system was developed that identifies genes coding for exported proteins. This system was modified for A. pleuropneumoniae. Gene fusions between the coding region of some heterologous signal sequence plus sequences from a normally exported protein and ′phoA may result in the expression of PhoA activity. PhoA+ colonies are indicative of fusion proteins derived from plasmids containing a foreign DNA insert harboring a promoter, a translational start site and a functional signal sequence.

When the A. pleuropneumoniae signal sequence library representing approximately 8250 individual colonies in the phoA-E. coli strain CC118 was screened for the blue colony phenotype on media containing XP, 95 colonies were found to be PhoA+. One plasmid (pl-25) from the PhoA+ colonies contained a 375-bp A. pleuropneumoniae insert that was relevant to our objectives. BLASTX analysis of this fragment displayed 36% identity and 57% similarity to the Rhizobium leguminosarum FhuD protein, amino acids 23 to 80 (of 301 total). It also showed 35% identity and 48% similarity to the E. coli FhuD protein, amino acids 28 to 80 (of 296 total). To obtain the full fhuD sequence on the chromosome of A. pleuropneumoniae serotype 1, oligonucleotide primers (F7, R7; Table 2) were designed from the 5′- and 3′-ends of the sequence in pl-25. These were paired with a primer (FA1; Table 2) based on a region of promoter sequences for fhuA from E. coli. A PCR product of 1063 bp was obtained with the primer pair FA1/R7. The deduced amino acids of this amplicon displayed an uninterrupted open reading frame (ORF) for some sequences coding for FhuD; the ORF had 27% identity to the protein and 44% homology to the gene in E. coli.

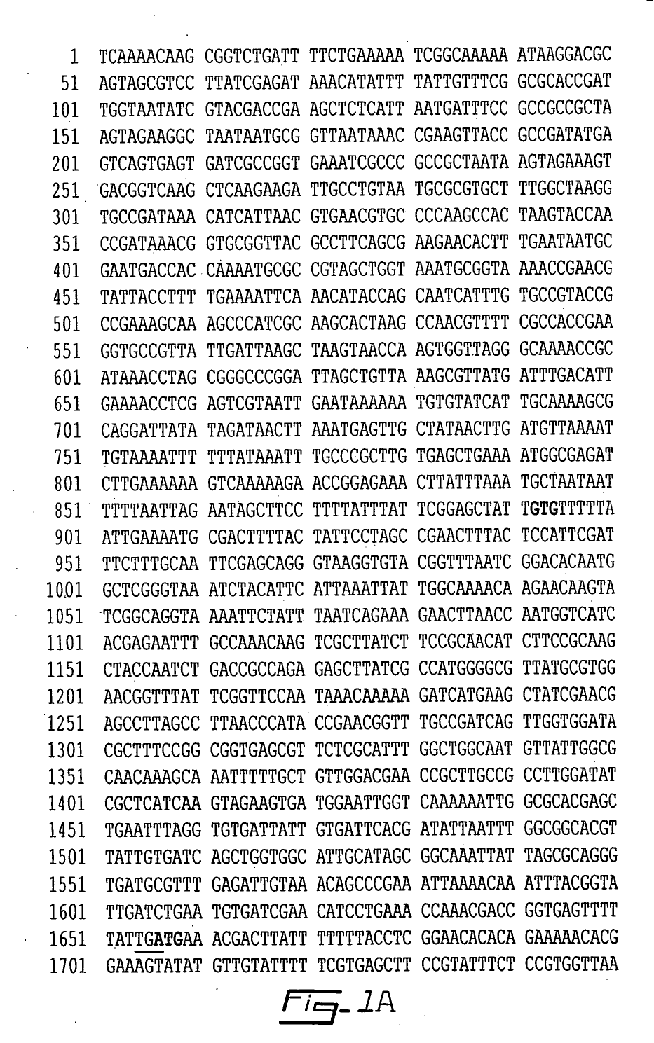

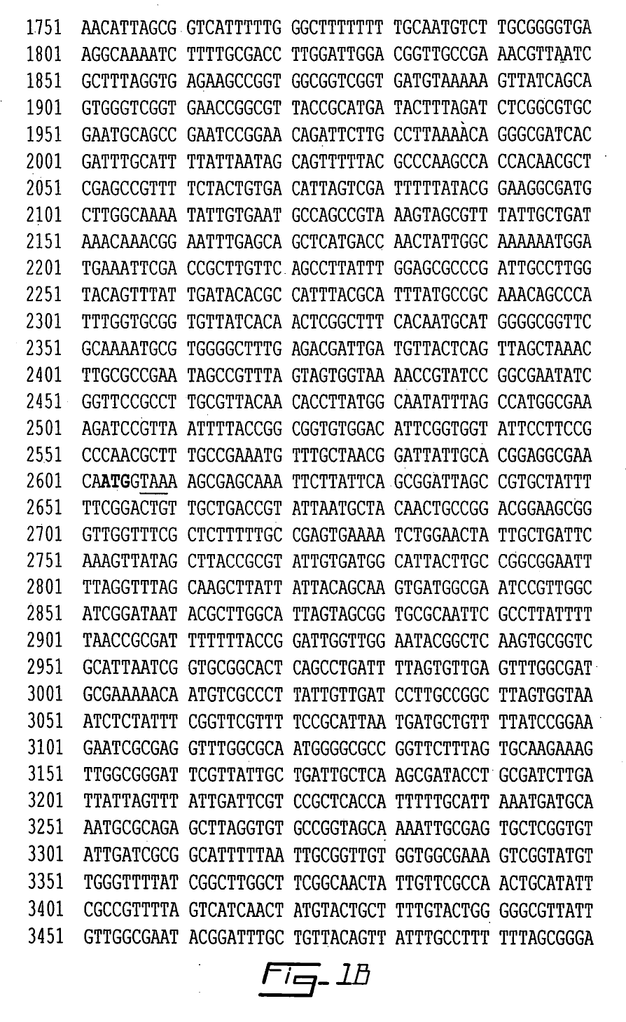

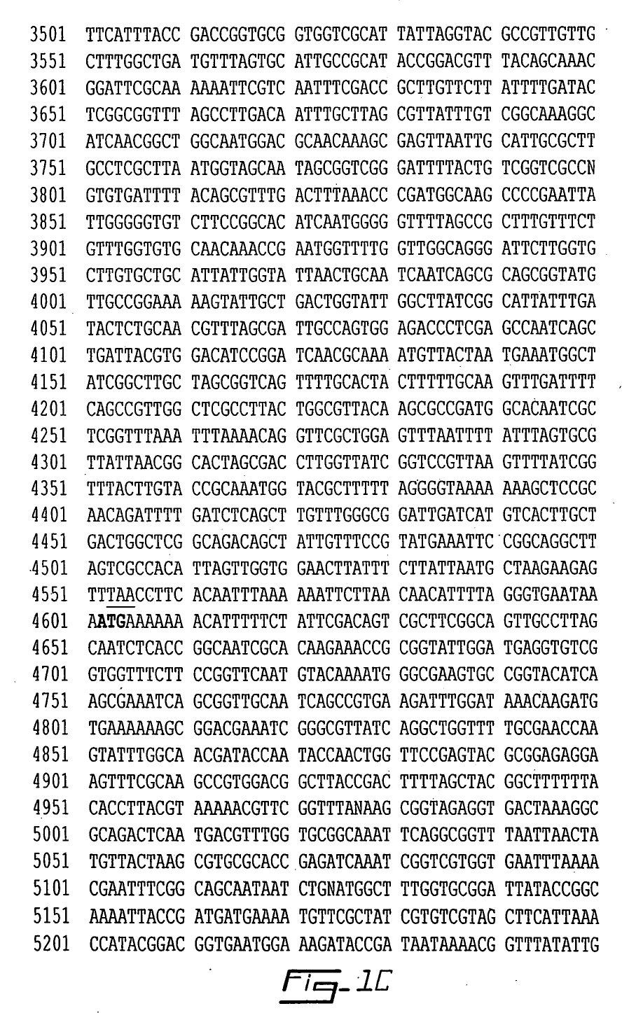

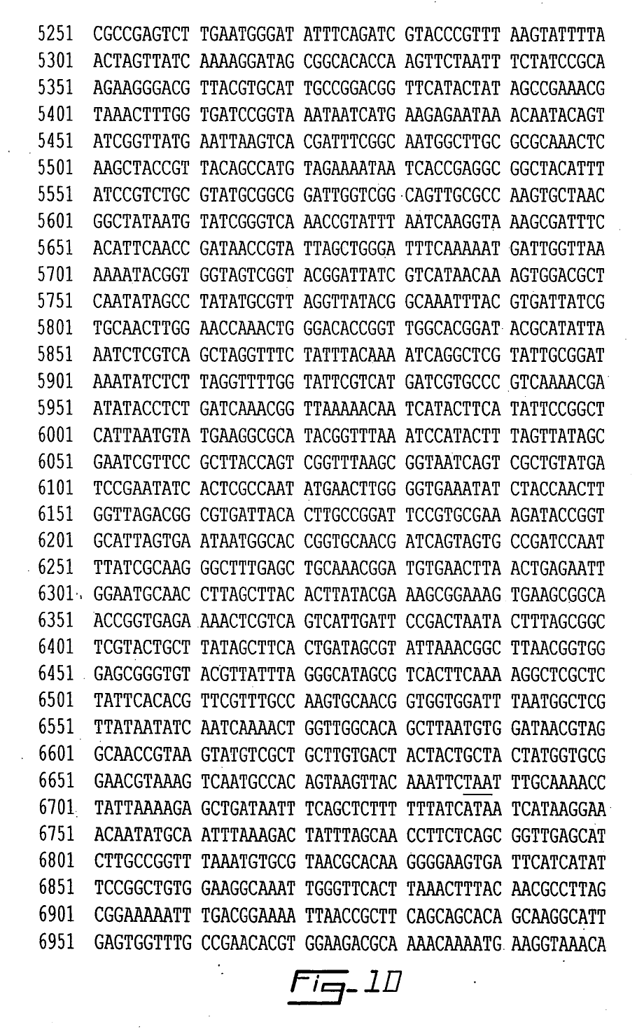

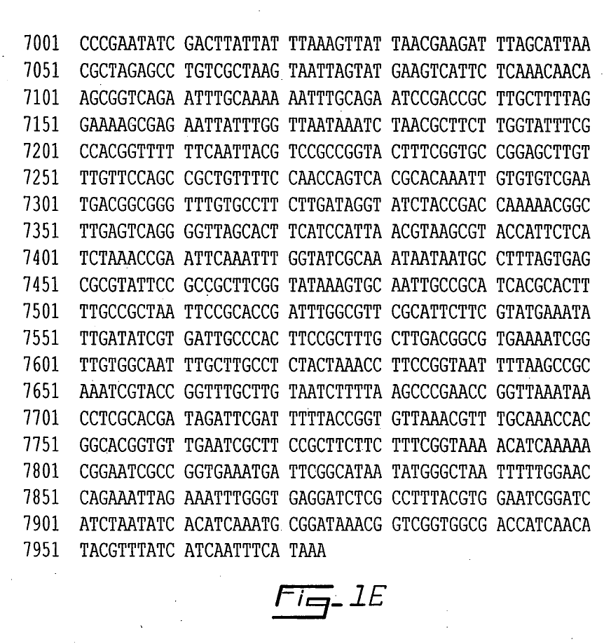

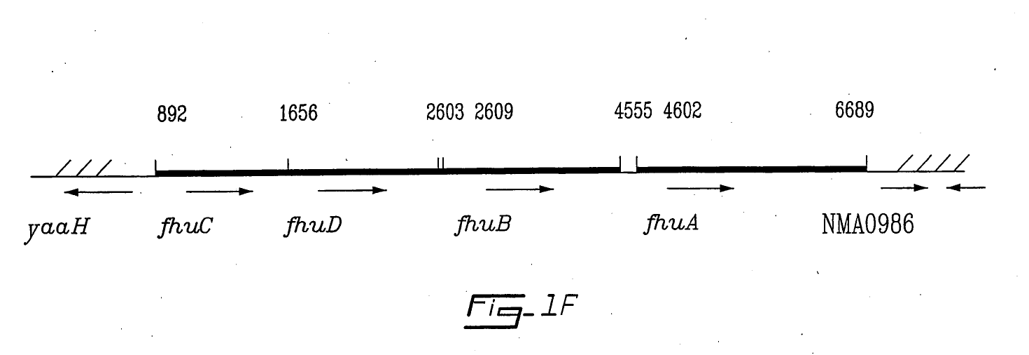

To isolate larger genomic fragments containing the fhuD gene with its neighboring sequences, a genomic library of A. pleuropneumoniae made in λZAP Express phage that contained fragments of A. pleuropneumoniae serotype 1 reference strain 4074 ranging approximately from 7- to 12-kbp was then used. As a screening tool, the 1063 bp PCR product (i.e. fhuD sequences) was labeled with digoxigenin. From ten plaques that gave a positive signal with the fhuD probe, one was subjected to three rounds of purification and the plasmid of this positive clone (pLM101) was then excised. Restriction analysis of this plasmid (FIG. 1D) revealed an insert of 8.0 kb. The entire insert was then sequenced, initially with universal primers T3 and T7 that annealed with the vector, and followed by sequencing with internal primers designed from the sequence walking. The total sequence information displayed four different ORFs and these corresponded to genes fhuC, fhuD, fhuB and fhuA in the fhu operon of several bacteria including E. coli.

Sequence of the fhuC Region

The nucleotide sequence coding for fhuC contains a single ORF extending from nucleotide 892 to 1656 (FIGS. 1A-C) which encodes a protein of 255 amino acids, predicted Mr of 28504 Da. The proposed translation start site is GTG (valine), known to act as a start codon in rare instances; the termination codon is a TGA. BLASTX analysis of this ORF displayed identity with the ATP-binding protein FhuC (53% E. coli, 47% R. leguminosarum, 41% Vibrio cholerae, 36% Bacillus subtilis). FhuC is a cytoplasmic protein belonging to the family of transporters that require binding proteins located in the periplasmic space. It is a hydrophilic but membrane-associated protein, containing two domains that are typical for nucleotide-binding proteins. Protein prediction analysis characterized the FhuC homologue in A. pleuropneumoniae as having an ATP/GTP binding site motif A (GHNGSGKS (SEQ ID NO:32), amino acids 34 to 41) and an ABC transporter family signature (LSGGERSRIWLAMLL (SEQ ID NO:33), amino acids 138 to 152).

Sequence of the FhuD Region.

In A. pleuropneumoniae, the last codon TGA of fhuC overlaps with the initiation codon ATG of the following gene fhuD which extends from nucleotide 1656 to 2609 (FIGS. 1A-C). This genetic organization matches the overlap of the fhuC and fhuD genes in E. coil. The nucleotide sequence of 954-bp displays an uninterrupted ORF terminating with a TAA and encodes a protein with a predicted Mr of 35656 Da. The deduced amino acid sequence of the ORF reveals a protein with identity to the FhuD of several bacteria (30% R. leguminosarum, 27% E. coli and 24% V. cholerae). FhuD is a periplasmic protein responsible for transporting ferrichrome from the FhuA receptor in the outer membrane to the FhuB protein in the cytoplasmic membrane. Using software for prediction of signal sequence cleavage sites, a possible cleavage site is proposed at amino acid 47 for FhuD of A. pleuropneumoniae; however the program failed to characterize this region as an N-terminal signal sequence that is usually displayed by proteins destined for export into the periplasm or outer membrane.

Sequence of the FhuB Region.

The initiation codon ATG for fhuB is situated 8-bp upstream of the termination codon TM of the preceding gene fhuD in the fhu operon of A. pleuropneumoniae. The ORF for FhuB, 651 amino acids, extends from nucleotide 2603 to 4555 (FIGS. 1A-C), terminating with a TM stop codon. The deduced amino acid sequence encodes a protein with a predicted Mr of 69370 Da displaying identity with the FhuB protein of several bacteria (40% Salmonella typhimurium, 39% R. leguminosarum, 37% E. coli and 35% V. cholerae). Software for prediction of protein localization identified the FhuB homologue in A. pleuropneumoniae as a cytoplasmic membrane protein with 19 membrane spanning regions compared to 16 membrane spanning regions that were predicted for E. coli. It was also characterized to have an ABC transporter family signature sequence (IASGDPRANQLITWT (SEQ ID NO:34), amino acids 489 to 503).

Sequence of the FhuA Region.

The last gene in the A. pleuropneumoniae fhu operon is an ORF from nucleotide 4602 to 6689 (FIGS. 1A-C) located 46-bp downstream of fhuB and terminating with a TAA codon. The deduced amino acid sequence of 696 amino acids encodes a protein with a predicted Mr of 77120 Da and it displays identity with the FhuA protein of several bacteria (31% V. cholerae, 27% P. aeruginosa, 26% E. coli, 26% S. typhimudium and 26% Enterobacter agglomerans). Software analysis for prediction of protein localization identified the putative A. pleuropneumoniae FhuA as an outer membrane protein having eleven extracellular loops and ten periplasmic turns as well as a cleavable N-terminal signal sequence which is characteristic of proteins destined for export to the outer membrane. A potential cleavage site for the signal sequence was detected between amino acid residues 23 and 24. The predicted molecular mass of the mature protein is 74750 Da. Clustal W multiple sequence alignment allowed comparison of the amino termini of the three known outer membrane proteins of A. pleuropneumoniae, TbpA, FhuA, and hemoglobin binding protein, HgbA. Within the first 13 amino acids of these three target sequences is located a stretch of six residues that may act as a TonB box:

| | | |
|---|---|---|
| TbpA | 1 EQAVQLNDVYVTG | (SEQ ID NO:35) |
| FhuA | 1 QETAVLDEVSVVS | (SEQ ID NO:36) |
| HgbA | 1 QEQMQLDTVIVKD | (SEQ ID NO:37) |

In other Gram-negative bacteria, the TonB box serves as a site of physical interaction between some outer membrane receptors and TonB, a protein that delivers the proton motive force of the cytoplasmic membrane to the outer membrane.

Characterization of the Fhu Chromosomal Locus in A. pleuropneumoniae Serotype 1

In contrast to the fhu operon in E. coli where fhuA is at the 5′-end, fhuA in A. pleuropneumoniae is preceded by the genes fhuC, fhuD and fhuB. Upstream of the fhuC gene in A. pleuropneumoniae is an incomplete ORF in the opposite direction, separated from the 5′-start of the fhuC gene by a 244-bp intergenic region and encoding a protein homologous to E. coli yaaH. This region contains promoter sites for the fhu genes as well as a Fur binding site for their regulation. A putative TAATTA (SEQ ID NO:38) box at −10 (nucleotides 854 to 858, FIGS. 1A-C) and a Shine-Dalgarno sequence (GGAG (SEQ ID NO:39); nucleotides 883 to 886, FIGS. 1A-C) were found in this stretch. A consensus sequence TTTAA (SEQ ID NO:40) for the −35 region (nucleotides 835 to 839, FIGS. 1A-C) was also identified. Both the −35 and Shine Dalgarno sequences match the consensus sequence proposed for promoter regions in A. pleuropneumoniae. The spacing between the −10 and −35 regions was 14 bp, a value which falls within the range of 13 and 16 bp proposed for this region in A. pleuropneumoniae as well. In E. coli, the fur gene product is a negative regulator of iron-dependent genes and the consensus sequence for the Fur box is GATAATGATAATCATTATC (SEQ ID NO:41). In A. pleuropneumoniae the region upstream of fhuC (nucleotides 834 to 853) and the stretch of 46 non-coding bp that falls between fhuB and fhuA (nucleotides 4598 to 4618) both demonstrated putative Fur boxes; 13/19 of the nucleotides were conserved upstream of fhuC and upstream of fhuA.

Analysis of DNA immediately downstream of A. pleuropneumoniae fhuA shows sequence homology with a hypothetical protein from Neisseria (NMA0986) in the same direction of transcription as the fhu genes. Four-hundred-bps downstream is a homologue to an H. influenzae protein (phospho-ribosyl-aminoamidazole-succinocarboxamide synthase) which is divergently transcribed.

Distribution of the Fhu Genes among A. pleuropneumoniae Serotype Reference Strains

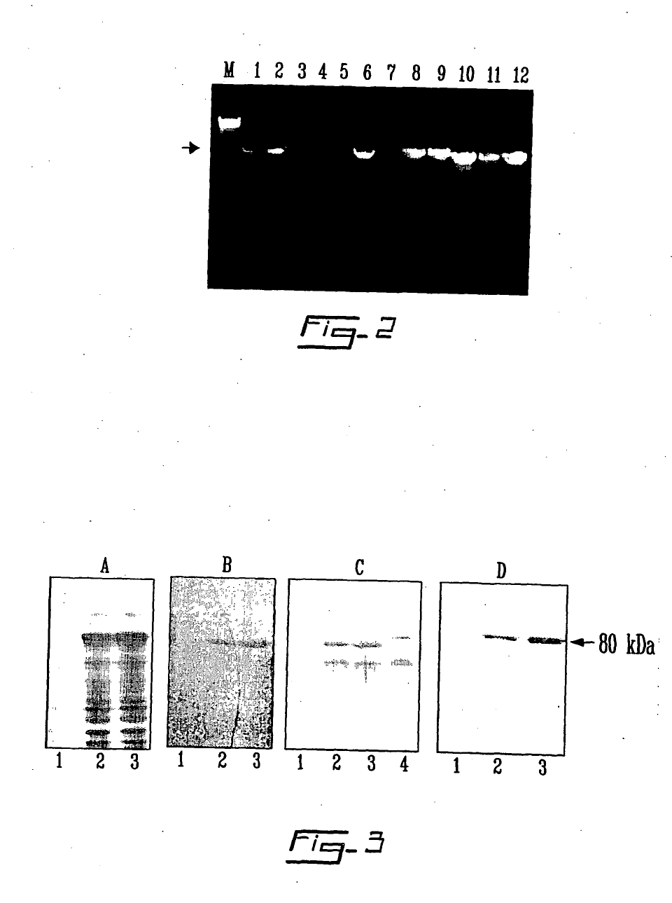

To determine whether the fhu operon is unique to A. pleuropneumoniae serotype 1 reference strain or is widely distributed, samples of DNA were investigated by PCR for reference strains representing the 11 other A. pleuropneumoniae serotypes. Primers 5T3W1 and 5T7W2 (Table 2) which flank the fhu region in A. pleuropneumoniae serotype 1 and the Expand Long Template PCR System 1 were used. Reference strains from serotypes 2, 6, 8, 9, 10, 11 and 12 all showed (FIG. 2) the expected 6.0 kb PCR product, identical to the one observed for serotype 1. Using primers (5T3W1 and 5T7W2; Table 2) flanking the fhu operon, some serotype strains yielded a negative PCR result for the entire fhu operon; these were further investigated. Amplification of the fhu operon in these serotypes was subsequently attempted with primers internal to the operon (CFor and ARev; Table 2). The expected PCR product of 5.7 kb was obtained for serotype 3, confirming that the fhu operon is present in this serotype and with an arrangement of fhu genes similar to that in serotype 1. However, the regions flanking the fhu operon are dissimilar in serotypes 1 and 3 as shown by the negative PCR result using primers flanking this region. Reference strains from serotypes 4, 5, and 7 were then investigated for the individual genes within the fhu operon both by PCR and Southern blot. For the PCR, pairs of primers internal to each gene were used: AFor/ARev for fhuA, BFor/BRev for fhuB, CFor/CRev for fhuC and DFor/DRev for fhuD (Table 2). For the Southern blot analyses, EcoRI-digested genomic DNA from these serotypes was tested for hybridization to a DIG-labeled PCR product for each gene. Reference strains from serotypes 4, 5 and 7 tested positive for fhuA, fhuB, fhuC and fhuD both by PCR and Southern blot although conditions of stringency had to be lowered to obtain a PCR amplicon and a positive hybridization signal for fhuD. This may be due to some dissimilarity of gene sequences between the fhuD of serotype 1 and serotypes 4, 5 and 7. Nevertheless, all genes of the fhu operon are present in all reference strains representing the known 12 serotypes of A. pleuropneumoniae.

Protein Expression and Western Blot

The fhuA of A. pleuropneumoniae serotype 1 was cloned into the expression vector pET30a to yield plasmid pLM202 (Table 1) and transformed into E. coli BL21 cells. The cells harboring the recombinant plasmid pLM202 were then grown under various conditions to optimize the expression of recombinant FhuA with the amino-terminal His-tag. Different concentrations of IPTG were tested and parameters of time and temperature were varied. Whole cell samples were run on 8% or 12.5% SDS-PAGE and the gels were stained with Coomassie Blue. A protein of approximately 79 kDa showed the highest level of expression at 25° C. (FIG. 3A). Induction with IPTG concentrations higher than 1.0 mM and induction periods longer than 6 h did not further enhance expression of recombinant FhuA. Gels were then transferred to a nitrocellulose membrane and blotted with an anti-His6 monoclonal antibody which reacted specifically with the 79 kDa protein: A. pleuropneumoniae FhuA with an N-terminal His-tag (FIG. 3B). Monoclonal antibody Fhu6.1 (FIG. 3 C) which recognizes a linear epitope between amino acids 241-281 of E. coli FhuA were then tested. For both Western blots, a purified FhuA.His6 of E. coli served as positive control, and uninduced E. coli BL21 cells harboring pLM202 served as negative control. Polyclonal antiserum of a pig experimentally infected with A. pleuropneumoniae serotype 1 and were able to show that the same protein of 79 kDa reacted also with the immune serum (FIG. 3, panel D) was also tested.

Homology Model for FhuA of A. pleuropneumoniae and Comparison with the Structure of FhuA of E. coli

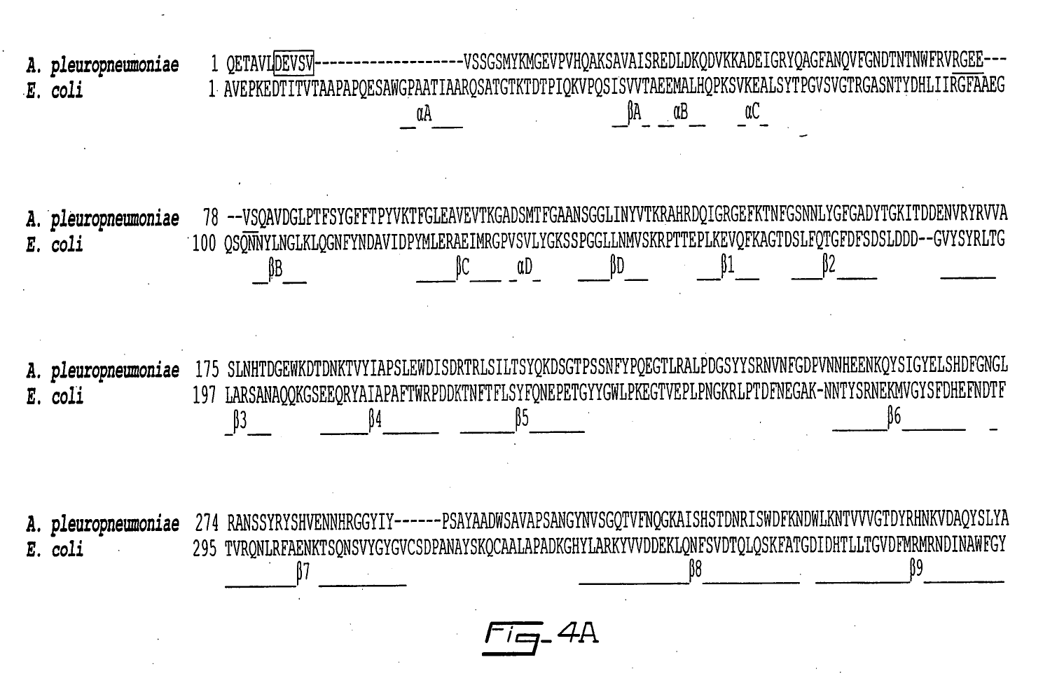

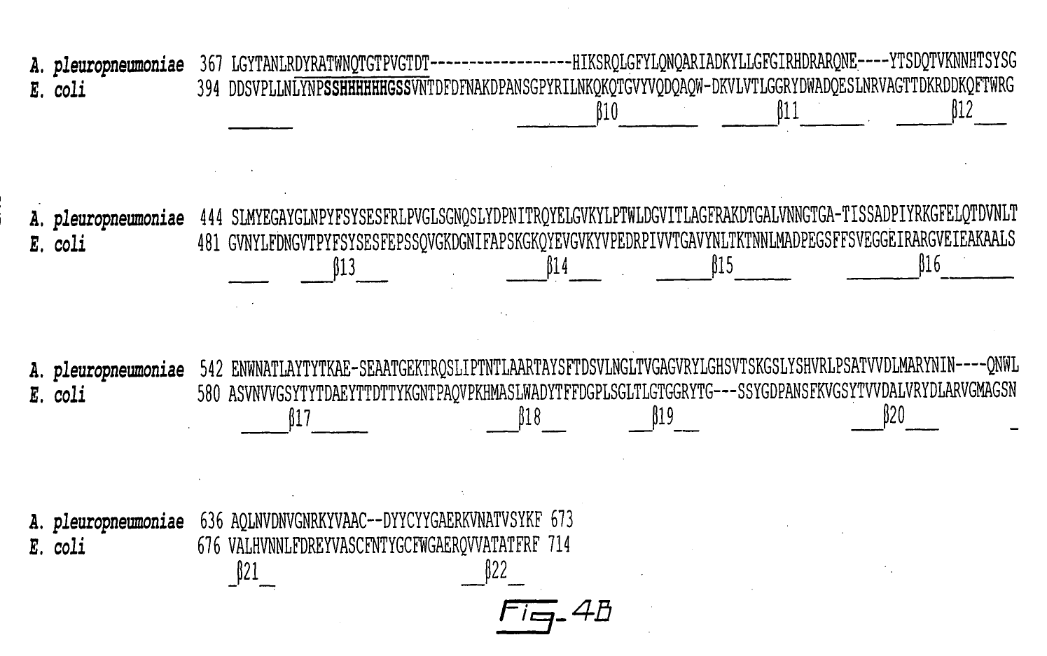

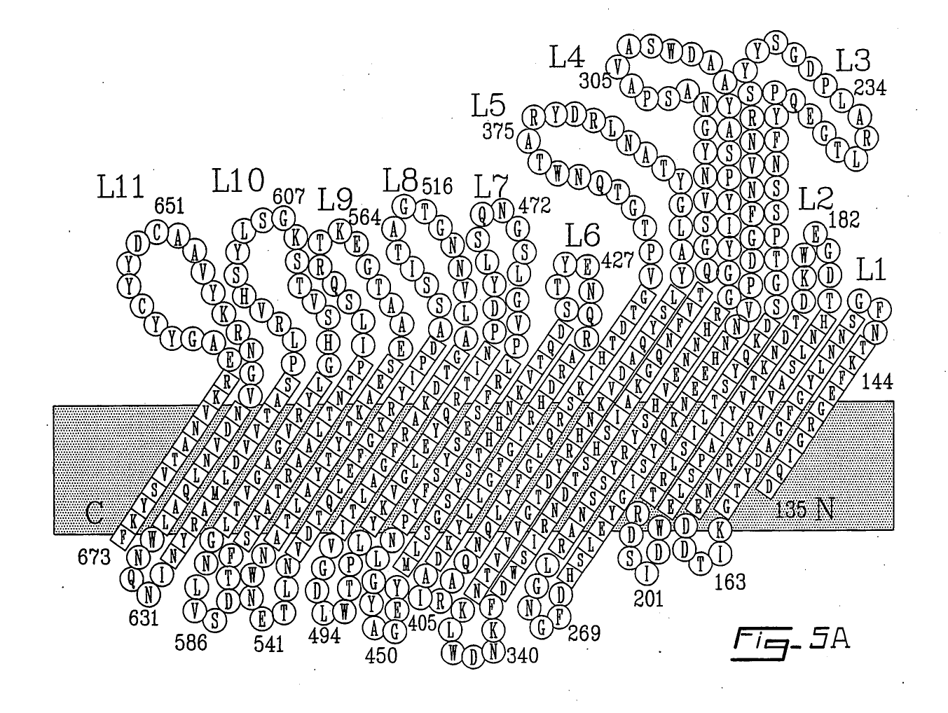

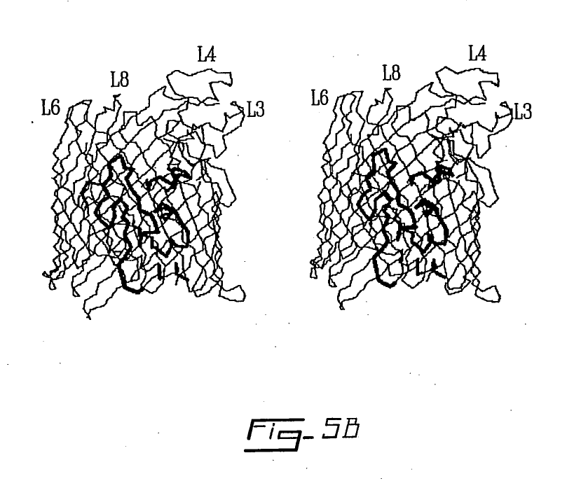

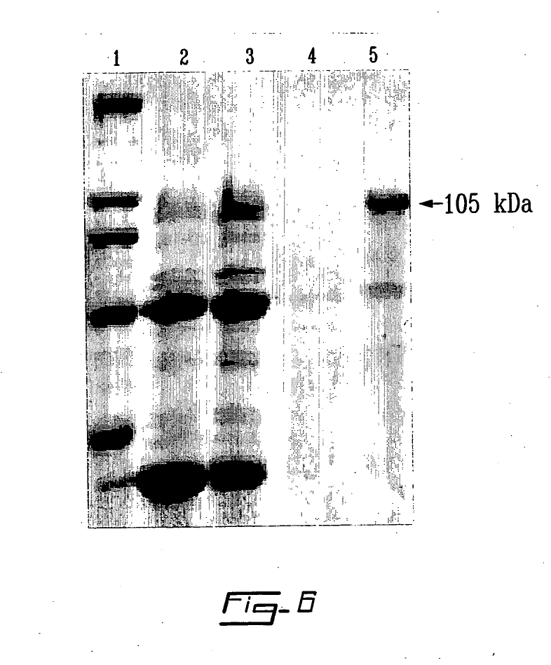

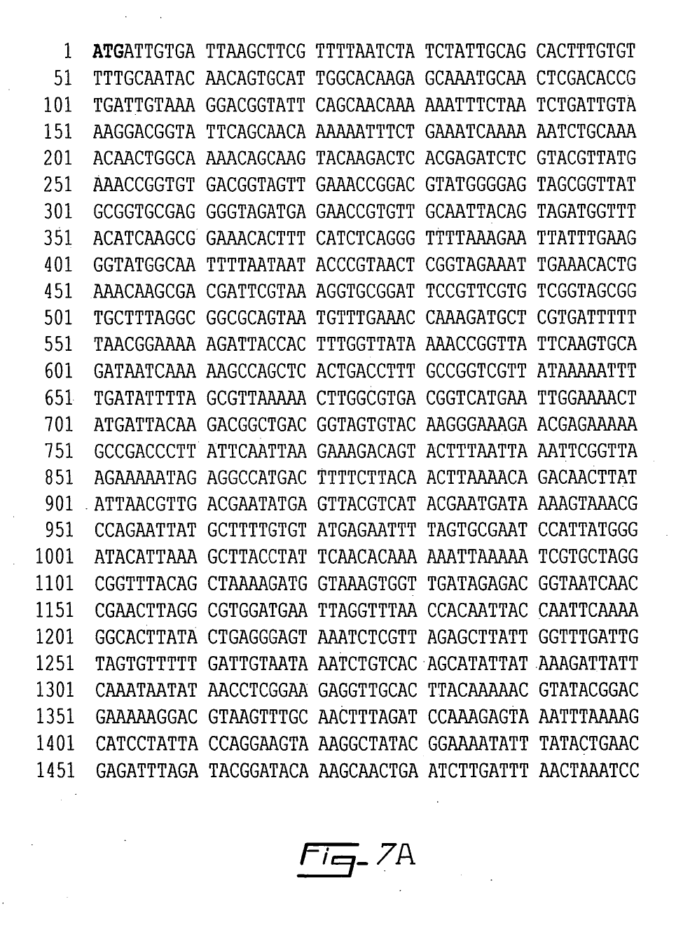

The JIGSAW and CODA servers were used to generate a composite homology model for FhuA of A. pleuropneumoniae. Following amino acid sequence alignment (FIG. 4) of FhuAs from A. pleuropneumoniae and E. coli, the model (FIG. 5A) for the A. pleuropneumoniae FhuA generated by the JIGSAW server showed eleven extracellular loops (L1-L11) and ten periplasmic turns. These numbers of extracellular loops and periplasmic turns are consistent with the E. coli FhuA structure, as is the presence of 22 β strands. FhuA of A. pleuropneumoniae shares the same overall fold as FhuA of E. coli. Both proteins possess two domains (FIG. 5B): an N-terminal cork domain (residues 1 to 134) in FhuA of A. pleuropneumoniae followed by a C-terminal β-barrel domain (residues 135 to 673) in FhuA of A. pleuropneumoniae. There were two gaps in the model: the first between residues 74-79 in the cork domain, and the other between residues 375-391 in the barrel domain. The second gap overlaps the hexahistidine tag and flanking linker regions (total of 11 amino acids) that were inserted to facilitate affinity purification of a E. coli recombinant FhuA. It is reasonable that the A. pleuropneumoniae model structure should not be threaded against this region since it is not present in the wild-type A. pleuropneumoniae fhuA gene. It was shown that residues 375 to 391 in the A. pleuropneumoniae FhuA structure is predominantly located within extracellular loop L5, with a short C-terminal stretch being contained within the adjacent β-strand (FIG. 5A).

Immunological Response

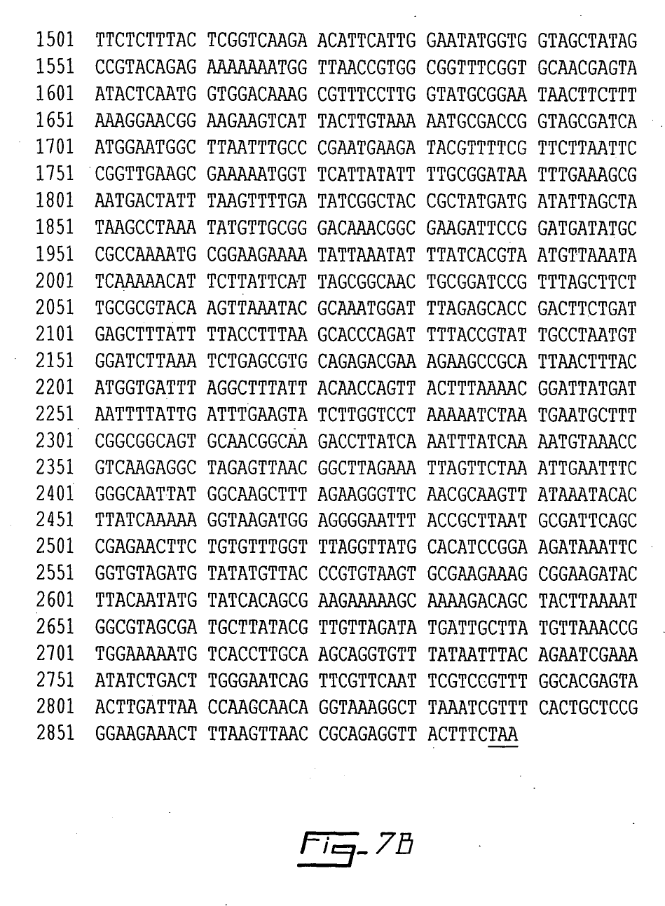

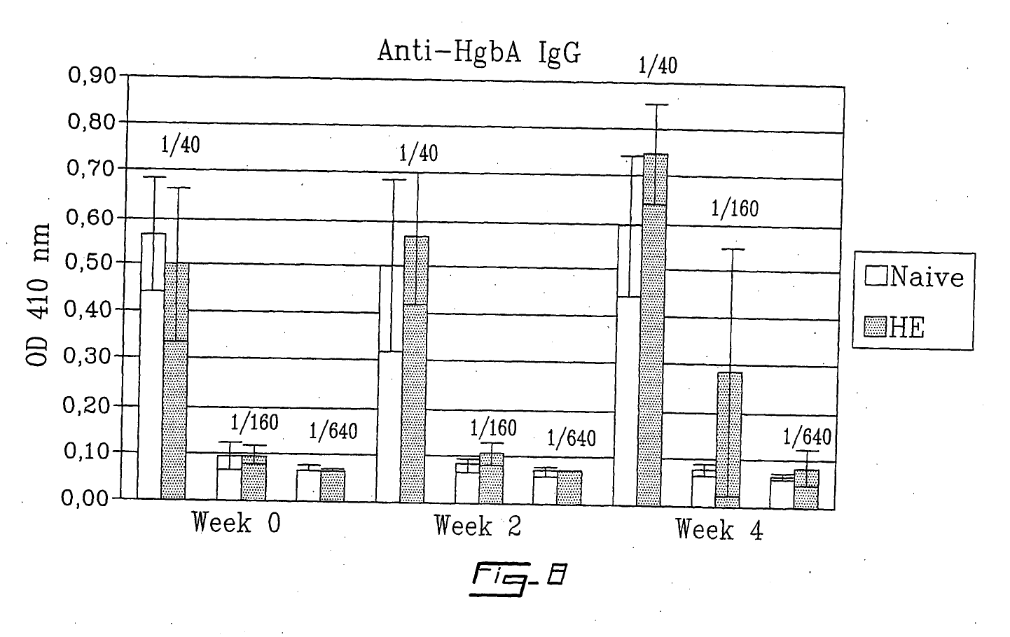

IgG levels against specific antigens become apparent (FIGS. 8 and 9) in the immunized groups 14 days after second immunization for both groups immunized intramuscularly (HE and FE). This is most evident in higher serum dilutions (1/160 and 1/640) where the IgG levels are significantly higher for animals that received FE and HE compared to the naïve animals.

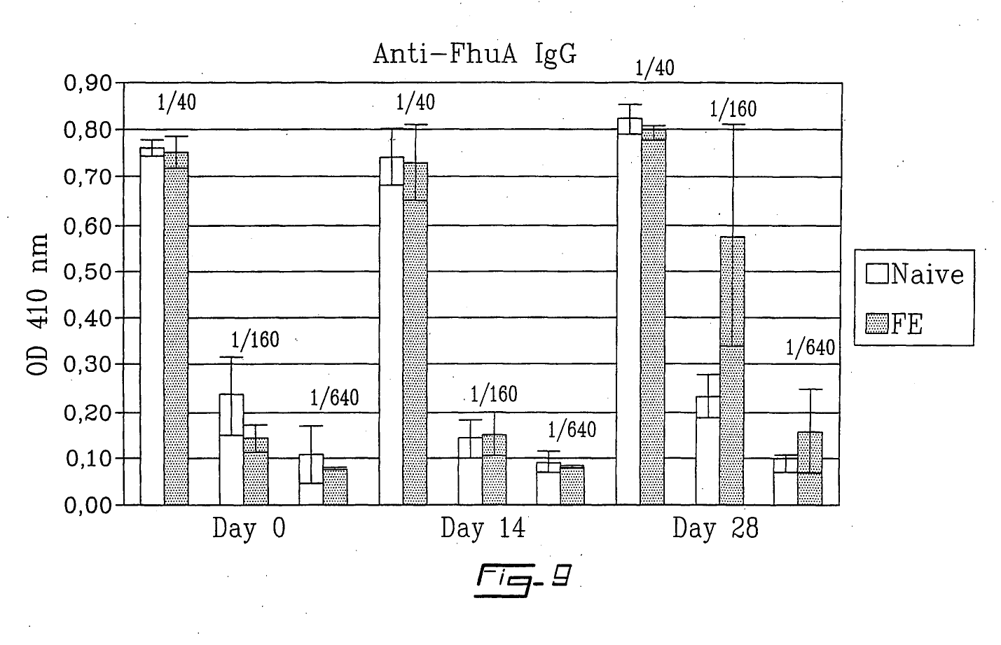

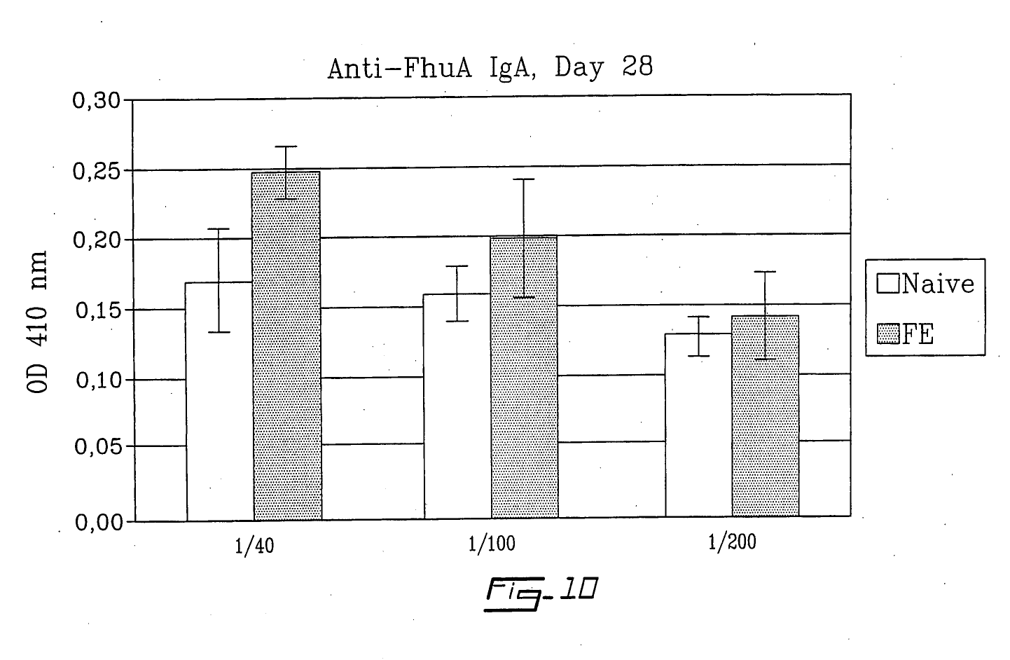

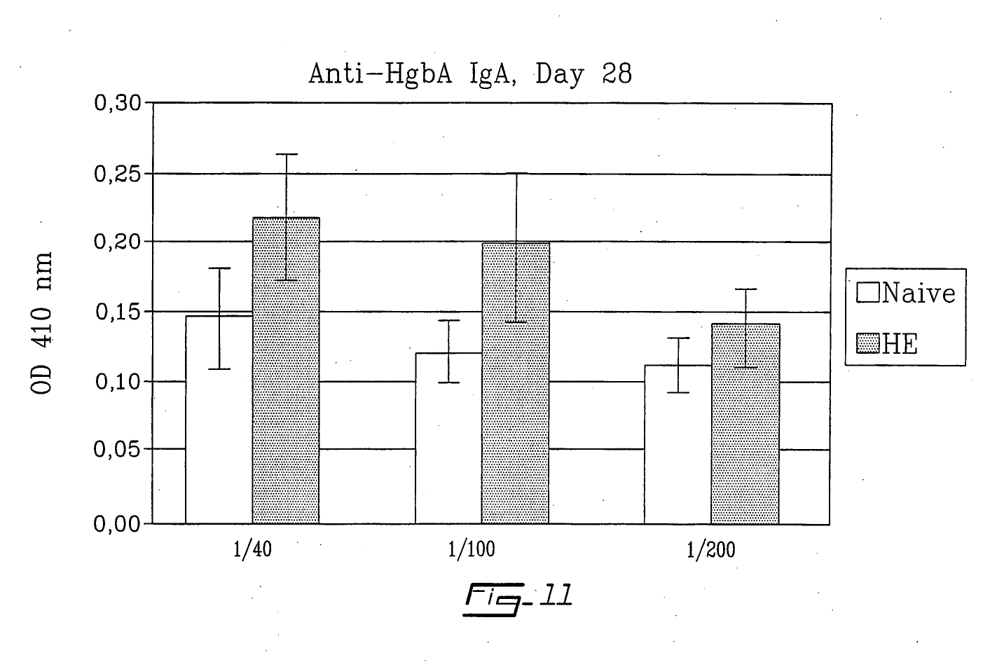

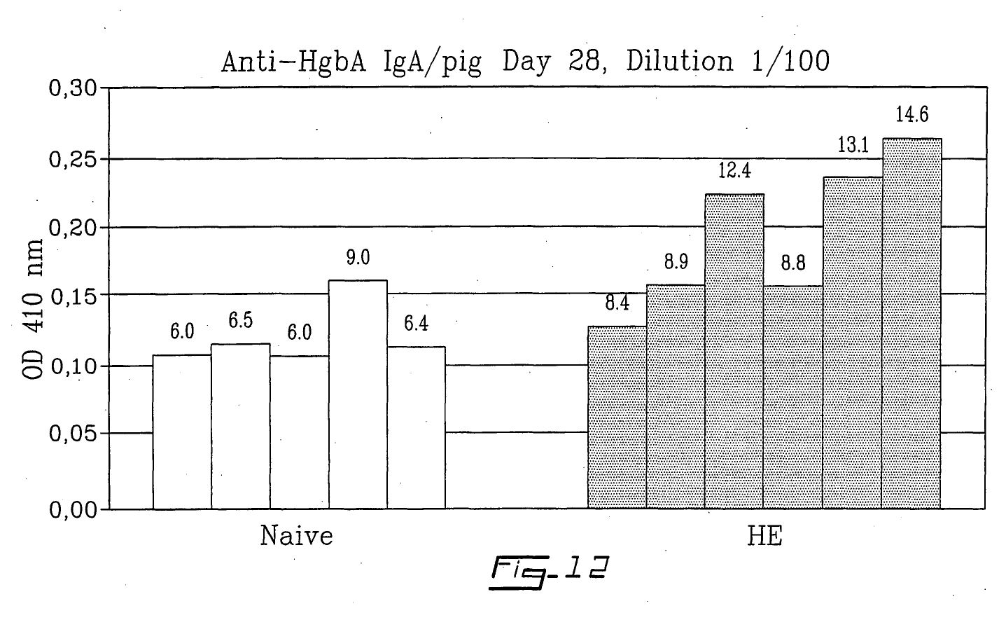

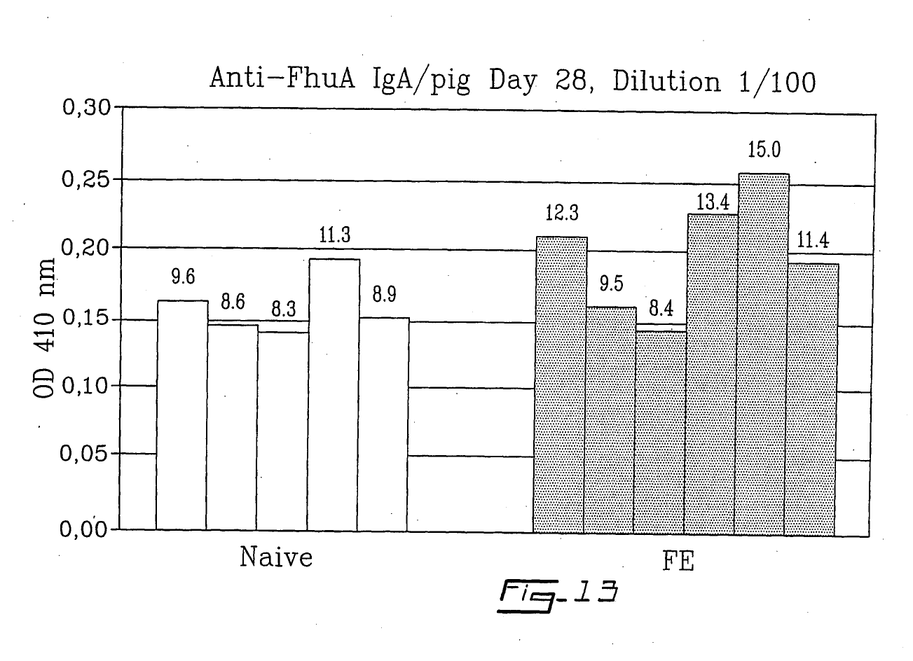

On the average, serum IgA levels (FIGS. 10 and 11) increase for the vaccinated groups (HE: 11.03 μg/ml and FE: average 11.66 μg/ml) compared to the naive groups (6.78 μg/ml and 9.34 μg/ml). Although the difference between vaccinated and naive groups is less than that observed for IgG levels, in the mammalian host, IgA represents less than 3% of total antibodies in the serum. Because of individual variation among pigs in each group (FIGS. 12 and 13), the values could reach up to three times higher than the naive lots for the immunized groups when individual pigs are monitored.

### DISCUSSION

In order to obtain the necessary structural information for analysis of outer membrane proteins, X-ray crystallography is used. The use of this technique has greatly expanded over the course of the last decade, allowing a number of crystal structures of outer membrane proteins to be solved, contributing to a major improvement in understanding the mechanisms underlying various bacterial processes, including nutrient uptake. In addition, a parallel objective of this application is the isolation and purification of the Hb-binding protein and the ferrichrome receptor of A. p. in order to use them as a vaccine against porcine pleuropneumoniae. A. p. is an important pathogen of swine, causing a hemorrhagic lung infection which is often lethal. Vaccine development also allows long-term protection from this disease.

These objectives are linked by their absolute requirement for isolation of the target protein in a pure form. Furthermore, this purification must be achieved at a relatively large scale, as both structural analysis and vaccine efficacy studies require milligram amounts of protein. Protein purification at this scale is most reproducibly accomplished with the use of fast-performance liquid chromatography (FPLC) which allows the passage of solutes through a column at high pressure. In light of these requirements, it would be advantageous to develop an immobilized-ligand affinity chromatography matrix amenable for use in a FPLC system as this type of matrix is commercially unavailable at the present time.

A Hb-binding protein from A. p. was identified through small-scale batch-binding experiments. Conditions for dissociation of the protein from its cognate ligand were assessed, and it was found that washing with high salt concentration was insufficient to disrupt the interaction. Subsequent analysis revealed that the use of a strong chaotropic agent, 2 M guanidine HCl, was able to elute a significant proportion of the Hb-binding protein, although some remained on the matrix, removed only by elution with SDS sample buffer. The removal of the protein is not improved with an increase in concentration of the chaotropic agent, as washing with 4 M guanidine-HCl still allowed approximately half of the Hb-binding protein to remain bound to the matrix. It should be noted that the use of chaotropic agents for elution may induce the denaturation of the protein of interest, which would necessitate further study of the process of its refolding.

Several conditions and matrices were screened in the process of achieving a stable complex of porcine Hb to pressure-stable matrices with the ability to withstand FPLC analysis. The choice of Affi-prep 10™ Affinity Support was made because of its high pressure stability and the broad range of ligands to which it is reported to efficiently couple. Verification by batch-binding revealed that use of 6 mg*ml⁻¹ porcine Hb in MOPS buffer of pH 5.5 or 6.5 yields a small amount of binding to the N-hydroxysuccinimide ester-activated matrix. The efficiency of this binding may be improved with further manipulation of binding conditions. CNBr-activated Sepharose 4B™ Fast Flow was selected on the basis of this product's ability to form stable linkages with many different ligands. The basis for stable binding is the product's ability to form multipoint attachment with the protein of interest, linking to accessible primary amines. The efficiency of binding, as measured by a protein dye-binding assay, was found to be approximately 8 mg*ml⁻of matrix. A batch-binding assay was also performed, but under the binding conditions used, it was shown that this matrix was unable to bind to the A. p. Hb-binding protein, whereas purification with the commercially-prepared Hb-agarose matrix (positive control) generated the expected 105 kDa band. This lack of binding may reflect of a change in conformation which occurs due to the multipoint binding of primary amines at the surface of Hb, perhaps altering the three dimensional shape of the moiety which is recognized by the binding protein, or perhaps hiding this moiety altogether. Further experimentation with binding conditions may help to maximize the process of purification of the Hb-binding protein using the laboratory-prepared matrix.

In-gel digestion of the protein of interest allowed analysis by QSTAR mass spectrometry. The determination of amino acid sequences for three internal peptides is currently being used in order to elucidate the DNA sequence of the gene encoding the Hb-binding protein. This information may be used for a number of purposes. First, the cloning of the coding gene into an appropriate expression vector may allow an increased expression of the protein either within A. p. cells, or within an artificial expression vector, such as E. coli. This may allow the purification of greater amounts of protein. In addition, it may be possible to clone the gene into an expression vector which possesses an additional feature to be used for purification, such as a PET vector, which allows the addition of a hexahistidine tag to the amino-terminal portion of the protein. The complete Hb-binding protein sequence may also be inputted into a computer modelling program which proposes likely three-dimensional strucures of OM β-barrel proteins on the basis of amino acid sequence; this modelling is done on the basis of sequence-structure relationships of similar proteins. This may allow the rational selection of a site at which a hexahistidine tag may be added to a surface-exposed loop, allowing for purification by metal chelate affinity chromatography.

Bacterial virulence is determined by the ability of an organism to compete for essential nutrients. Because the mammalian host restricts bacterial growth by withholding iron, most bacteria have evolved a diverse series of high affinity iron acquisition systems to satisfy their iron requirements. One such system is the synthesis and/or uptake of low molecular weight iron chelators termed siderophores. Transporters bind these iron chelates with high affinity and mediate their uptake across the outer membrane of Gram-negative bacteria. The proteins required for the uptake of ferric hydroxamates are the products of the genes fhuA, fhuC, fhuD and fhuB, where FhuA acts as the receptor in the outer membrane for ferrichrome and whose crystal structure has recently been determined. The energy required to translocate these compounds is derived from the proton motive force of the cytoplasmic membrane as transduced by the TonB-ExbB-ExbD complex.

A. pleuropneumoniae, a Gram-negative bacterium that is an important swine pathogen, is capable of using transferrin, hemoglobin, hemin and exogenous siderophores including ferric hydroxamates as sources of iron for growth. The transferrin receptor complex includes TbpA and TbpB in the outer membrane of A. pleuropneumoniae, as well as the exbB and exbD genes upstream of tbpA and tbpB in the same operon. One of the objectives of the present application was to elucidate the genes and their products involved in the uptake of ferric hydroxamates in A. pleuropneumoniae. The homologs of the E. coli fhuACDB operon were successfully cloned from A. pleuropneumonia. The fhuA gene encodes the 77 kDa outer membrane protein FhuA acting as the receptor for ferric hydroxamate; FhuD is the periplasmic protein responsible for the translocation of ferric hydroxamate from the outer to the inner membrane; FhuC and FhuB are cytoplasmic membrane-associated proteins, components of an ABC transporter which internalize the ferric hydroxamate. An ABC transporter family signature sequence was identified for both FhuC and FhuB of A. pleuropneumoniae. In other Gram-negative bacteria, FhuB is a hydrophobic protein embedded in the cytoplasmic membrane. Southern blot and PCR analyses showed that all the genes of the fhu operon are present in all reference strains representing the known 12 serotypes of A. pleuropneumoniae in the same order.

The genes for the uptake of ferrichrome are arranged differently in A. pleuropneumoniae than the corresponding genes in E. coli. In the latter, the ferric hydroxamate uptake receptor gene fhuA is located upstream of fhuCDB whereas in A. pleuropneumoniae it is the last gene transcribed in the fhu operon. This difference in gene organization between A. pleuropneumoniae and E. coli may reflect differences in the regulation of these two iron transport systems. To explore this possibility, further analyses are needed on the sequences upstream of fhuC and fhuA in A. pleuropneumoniae. The G+C content of the fhu operon in A. pleuropneumoniae is 44%, a value that correlates well with the estimated 43.2% reported for the genome of A. pleuropneumoniae.

The predicted primary sequence of FhuA from A. pleuropneumoniae has allowed us to propose a homology-based three-dimensional model of the protein (FIG. 5B). The overall fold of this model resembles that of E. coli FhuA for which the crystal structure is known, with the most significant deviations from the known structure occurring in the extracellular and periplasmic loop regions. Overall, the sizes of the extracellular loops of the FhuA model are similar to the corresponding loops in the E. coli structure. However, significant differences between the two lengths of key extracellular loop regions were observed. Loop L3 in the E. coli structure is 31 residues compared to 35 residues for L3 in the model of FhuA from A. pleuropneumoniae due to an extension of the loop at its N-terminal end. Furthermore, L4 in the model of FhuA from A. pleuropneumoniae is considerably longer (28 residues compared to 20 residues in the E. coli L4 loop) and does not contain the short beta strands that are seen in the E. coli FhuA structure. Given that loops L3 and L4 are involved in ligand recognition and uptake, it remains to be determined if these structural variations correspond to differences in function of FhuA from A. pleuropneumoniae relative to the E. coli protein. These differences could also prove to be responsible for the lack of susceptibility of A. pleuropneumoniae to the antibiotics albomycin [check] and rifamycin CGP 4832 and the bacterial toxin colicin M, all of which use FhuA as docking sites for entry into E. coli bacterial cells.

The x-ray crystallographic structure of E. coli FhuA complexed with ferricrocin indicates that ten residues are within 4 Å of the bound ligand atoms. Inspection of the structure-based alignment of A. pleuropneumoniae FhuA with the primary sequence of the E. coli protein (FIG. 4) shows that six of ten positions are highly homologous. Absolute conservation is observed with residues Y292 and Y294, which align with the E. coli ligand-binding residues Y313 and Y315. Residues F92 and F224 share the aromatic character of the aligned E. coli residues Y116 and W246. A position of hydrophobicity is maintained at position L364 which aligns with F391 in the E. coli sequence. Finally, S232, which has the potential to hydrogen bond to the siderophore ligand, is aligned with the E. coli residue Y244. Interestingly, two of these six residues retain an aromatic character relative to their homologous E. coli ligand-binding residues, yet they do not have the ability to form hydrogen bonds with a siderophore ligand. This suggests that either the mode of ligand binding for the A. pleuropneumoniae protein may have hydrophobic stacking interactions as a more predominant component, or it may indicate that the hydrophobic component of these residues in the E. coli protein may be more critical to ligand binding than their ability to form hydrogen bonds. Functional analysis of the ligand-binding ability of a mutant E. coli FhuA protein in which these residues have been modified to resemble the A. pleuropneumoniae sequence (Y116F, W246F) may resolve these possibilities.

The structure of E. coli FhuA also possesses a stretch of highly conserved residues along the longitudinal axis of the inner wall of the barrel that are thought to form a ‘staircase’, potentially to facilitate siderophore transfer from the ligand binding site to the periplasm. Of these eight staircase residues, four homologous positions are conserved in the A. pleuropneumoniae primary sequence as indicated by the structure-based alignment (FIG. 4). With a two-residue shift towards the C-terminus, R274 and N276 align with the E. coli residues R297 and N299, respectively. Absolute conservation is seen with two charged residues, D331 and D352, which align with D358 and D379 in the E. coli sequence. Finally, the position Q397 aligns perfectly with the E. coli staircase residue Q431. In the A. pleuropneumoniae sequence, R333 aligns with the E. coli staircase residue Q360. Although at this position there is a charge difference between the two proteins, both side-chains are of approximately the same length, suggesting that the role of this staircase residue may be based more on the steric character of the side-chain than its charge. It is interesting to note that with the exception of the Q397/Q431 pair, the highest degree of homology is clustered at the N-terminal end of the staircase, proximal to the periplasmic end of the barrel domain.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A DNA sequence of outer membrane proteins of Actinobacillus pleuropneumoniae expressed in a subject suffering from pleuropneumonia, said sequence coding for the receptor for the uptake of ferric hydroxamate FhuA as set forth in SED ID NO: 1 in Actinobacillus pleuropneumoniae, or any functional fragment thereof.

2. A DNA sequence of outer membrane proteins of Actinobacillus pleuropneumoniae expressed in a subject suffering from pleuropneumonia, said sequence coding for a hemoglobin-binding protein as set forth in SEQ ID NO: 4 in Actinobacillus pleuropneumoniae, or any functional fragment thereof.

3. A DNA sequence according to claims 1 or 2, wherein said subject is a pig.

4. A DNA sequence according to claims 1 or 2, wherein said sequence is present in all serotypes of A. pleuropneumoniae.

5. A DNA construct comprising an expression vector comprising the sequence of claim 1.

6. The construct of claim 5, wherein said expression vector is pLM202.

7. A DNA construct comprising an expression vector comprising the sequence of claim 2.

8. The construct of claim 7, wherein said expression vector is pRSC06.

9. A host cell stably transformed with the construct of claims 7 or 8.

10. The cell of claim 9, wherein said host is E. coli.

11. A recombinant protein obtained from the cell of claim 9.

12. A method for obtaining recombinant protein from the host cell of claim 9, comprising the steps of:
purifying proteins produced by the host cell of claim 9; and
resuspending said proteins in a detergent.

13. The method of claim 12, wherein purification is performed with Ni-NTA column.

14. The method as in claim 10 or 11, wherein resuspension is performed in a detergent selected from the group consisting of Guanidine hydrochoride, urea and Empigen.

15. A pharmaceutical composition for immunoprotecting a subject against Actinobacillus pleuropneumoniae, which comprises an amount of antigen encoded by the DNA sequence of any one of claims 1 or 2 sufficient to elicit a protecting immune response from said subject in association with a pharmaceutical acceptable carrier.

16. The composition of claim 15, wherein said carrier is for administration by a route selected from the group consisting of parenteral, oral, rectal, intranasal, topical, by instillation into the eye and by inhalation of an aerosol.

17. The composition of claim 15, wherein said carrier is for administration by intranasal route.

18. The use of the pharmaceutial composition of claim 15 as a vaccine against porcine pleuropneumonia.

19. An ELISA diagnostic kit for the assay of Actinobacillus pleuropneumoniae antibodies in a biological sample of pigs, comprising, at least one of the following:
a) a plate having bound thereto a purified A. pleuropneumoniae antigen for binding to anti-A. pleuropneumoniae antibodies present in the biological sample of pigs;
b) a positive control vial of biological sample from pigs experimentally inoculated with a strain of A. pleuropneumoniae;
c) a negative control vial of pig biological sample from A. pleuropneumonia free herd; and
d) a detectably labeled conjugate which binds to pig antibodies bound to the plate of a).

20. The kit of claim 19, wherein said biological sample is serum.

21. The kit of claim 19, wherein said detectably labeled conjugate is an anti-pig antibodies, which is directly or indirectly detected.

22. The kit of claim 21, further comprising a substrate which allow the visualization of the detectably labeled conjugate.

23. The kit of claim 22, wherein said detectably labeled conjugate comprises an enzyme label.

24. The kit of claim 23, wherein said substrate is a composition for providing a colorimetric, fluorimetric orchemiluminescent signal in the presence of said enzyme label.

25. The kit as in one of claims 19-24, wherein said kit is suitable for any Actinobacillus pleuropneumoniae serotypes.

26. A method of diagnosis of A. pleuropneumonia infection in a pig biological sample, which comprises the steps of:
a) subjecting an ELISA plate having bound thereto an antigen encoded by the DNA sequence of any one of claims 1 and 2 with said pig biological sample; and
b) detecting the presence of A. pleuropneumonia antibodies in said sample; whereby the presence of antibodies is indicative of a A. pleuropneumonia infection.

27. The method of claim 26, wherein said biological sample is serum.


SEQUENCE LISTING (41 sequences)

SEQ ID NO: 1 - DNA, 7974 bp, Artificial Sequence
  fhu operon in Actinobacillus pleuropneumoniae
tcaaaacaagcggtctgattttctgaaaaatcggcaaaaaataaggacgcagtagcgtccttatcgagataaacatattttattgtttcggcgcaccgattggtaatatcgtacgaccgaagctctcattaatgatttccgccgccgctaagtagaaggctaataatgcggttaataaaccgaagttaccgccgatatgagtcagtgagtgatcgccggtgaaatcgcccgccgctaataagtagaaagtgacggtcaagctcaagaagattgcctgtaatgcgcgtgctttggctaaggtgccgataaacatcattaacgtgaacgtgccccaagccactaagtaccaaccgataaacggtgcggttacgccttcagcgaagaacactttgaataatgcgaatgaccaccaaaatgcgccgtagctggtaaatgcggtaaaaccgaacgtattaccttttgaaaattcaaacataccagcaatcatttgtgccgtaccgccgaaagcaaagcccatcgcaagcactaagccaacgttttcgccaccgaaggtgccgttattgattaagctaagtaaccaagtggttagggcaaaaccgcataaacctagcgggcccggattagctgttaaagcgttatgatttgacattgaaaacctcgagtcgtaattgaataaaaaatgtgtatcattgcaaaagcgcaggattatatagataacttaaatgagttgctataacttgatgttaaaattgtaaaattttttataaatttgcccgcttgtgagctgaaaatggcgagatcttgaaaaaagtcaaaaagaaccggagaaacttatttaaatgctaataatttttaattagaatagcttccttttatttattcggagctattgtgtttttaattgaaaatgcgacttttactattcctagccgaactttactccattcgatttctttgcaattcgagcagggtaaggtgtacggtttaatcggacacaatggctcgggtaaatctacattcattaaattattggcaaaacaagaacaagtatcggcaggtaaaattctatttaatcagaaagaacttaaccaatggtcatcacgagaatttgccaaacaagtcgcttatcttccgcaacatcttccgcaagctaccaatctgaccgccagagagcttatcgccatggggcgttatgcgtggaacggtttattcggttccaataaacaaaaagatcatgaagctatcgaacgagccttagccttaacccataccgaacggtttgccgatcagttggtggatacgctttccggcggtgagcgttctcgcatttggctggcaatgttattggcgcaacaaagcaaatttttgctgttggacgaaccgcttgccgccttggatatcgctcatcaagtagaagtgatggaattggtcaaaaaattggcgcacgagctgaatttaggtgtgattattgtgattcacgatattaatttggcggcacgttattgtgatcagctggtggcattgcatagcggcaaattattagcgcagggtgatgcgtttgagattgtaaacagcccgaaattaaaacaaatttacggtattgatctgaatgtgatcgaacatcctgaaaccaaacgaccggtgagtttttattgatgaaacgacttatttttttacctcggaacacacagaaaaacacggaaagtatatgttgtatttttcgtgagcttccgtatttctccgtggttaaaacattagcggtcatttttgggcttttttttgcaatgtcttgcggggtgaaggcaaaatcttttgcgaccttggattggacggttgccgaaacgttaatcgctttaggtgagaagccggtggcggtcggtgatgtaaaaagttatcagcagtgggtcggtgaaccggcgttaccgcatgatactttagatctcggcgtgcgaatgcagccgaatccggaacagattcttgccttaaaacagggcgatcacgatttgcattttattaatagcagtttttacgcccaagccaccacaacgctcgagccgttttctactgtgacattagtcgatttttatacggaaggcgatgcttggcaaaatattgtgaatgccagccgtaaagtagcgtttattgctgataaacaaacggaatttgagcagctcatgaccaactattggcaaaaaatggatgaaattcgaccgcttgttcagccttatttggagcgcccgattgccttggtacagtttattgatacacgccatttacgcatttatgccgcaaacagcccatttggtgcggtgttatcacaactcggctttcacaatgcatggggcggttcgcaaaatgcgtggggctttgagacgattgatgttactcagttagctaaacttgcgccgaatagccgtttagtagtggtaaaaccgtatccggcgaatatcggttccgccttgcgttacaacaccttatggcaatatttagccatggcgaaagatccgttaattttaccggcggtgtggacattcggtggtattccttccgcccaacgctttgccgaaatgtttgctaacggattattgcacggaggcgaacaatggtaaaagcgagcaaattcttattcagcggattagccgtgctatttttcggactgttgctgaccgtattaatgctacaactgccggacggaagcgggttggtttcgctctttttgccgagtgaaaatctggaactattgctgattcaaagttatagcttaccgcgtattgtgatggcattacttgccggcggaattttaggtttagcaagcttattattacagcaagtgatggcgaatccgttggcatcggataatacgcttggcattagtagcggtgcgcaattcgccttatttttaaccgcgatttttttaccggattggttggaatacggctcaagtgcggtcgcattaatcggtgcggcactcagcctgattttagtgttgagtttggcgatgcgaaaaacaatgtcgcccttattgttgatccttgccggcttagtggtaaatctctatttcggttcgttttccgcattaatgatgctgttttatccggaagaatcgcgaggtttggcgcaatggggcgccggttctttagtgcaagaaagttggcgggattcgttattgctgattgctcaagcgatacctgcgatcttgattattagtttattgattcgtccgctcaccatttttgcattaaatgatgcaaatgcgcagagcttaggtgtgccggtagcaaaattgcgagtgctcggtgtattgatcgcggcatttttaattgcggttgtggtggcgaaagtcggtatgttgggttttatcggcttggcttcggcaactattgttcgccaactgcatattcgccgttttagtcatcaactatgtactgcttttgtactgggggcgttattgttggcgaatacggatttgctgttacagttatttgccttttttagcgggattcatttaccgaccggtgcggtggtcgcattattaggtacgccgttgttgctttggctgatgtttagtgcattgccgcataccggacgtttacagcaaacggattcgcaaaaaattcgtcaatttcgaccgcttgttcttattttgatactcggcggtttagccttgacaatttgcttagcgttatttgtcggcaaaggcatcaacggctggcaatggacgcaacaaagcgagttaattgcattgcgcttgcctcgcttaatggtagcaatagcggtcgggattttactgtcggtcgccngtgtgattttacagcgtttgactttaaacccgatggcaagccccgaattattgggggtgtcttccggcacatcaatgggggttttagccgctttgtttctgtttggtgtgcaacaaaccgaatggttttggttggcagggattcttggtgcttgtgctgcattattggtattaactgcaatcaatcagcgcagcggtatgttgccggaaaaagtattgctgactggtattggcttatcggcattatttgatactctgcaacgtttagcgattgccagtggagaccctcgagccaatcagctgattacgtggacatccggatcaacgcaaaatgttactaatgaaatggctatcggcttgctagcggtcagttttgcactactttttgcaagtttgattttcagccgttggctcgccttactggcgttacaagcgccgatggcacaatcgctcggtttaaatttaaaacaggttcgctggagtttaattttatttagtgcgttattaacggcactagcgaccttggttatcggtccgttaagttttatcggtttacttgtaccgcaaatggtacgctttttaggggtaaaaaaagctccgcaacagattttgatctcagcttgtttgggcggattgatcatgtcacttgctgactggctcggcagacagctattgtttccgtatgaaattccggcaggcttagtcgccacattagttggtggaacttatttcttattaatgctaagaagagtttaaccttcacaatttaaaaaattcttaacaacattttagggtgaataaaatgaaaaaaacatttttctattcgacagtcgcttcggcagttgccttagcaatctcaccggcaatcgcacaagaaaccgcggtattggatgaggtgtcggtggtttcttccggttcaatgtacaaaatgggcgaagtgccggtacatcaagcgaaatcagcggttgcaatcagccgtgaagatttggataaacaagatgtgaaaaaagcggacgaaatcgggcgttatcaggctggttttgcgaaccaagtatttggcaacgataccaataccaactggttccgagtacgcggagaggaagtttcgcaagccgtggacggcttaccgacttttagctacggcttttttacaccttacgtaaaaacgttcggtttanaagcggtagaggtgactaaaggcgcagactcaatgacgtttggtgcggcaaattcaggcggtttaattaactatgttactaagcgtgcgcaccgagatcaaatcggtcgtggtgaatttaaaacgaatttcggcagcaataatctgnatggctttggtgcggattataccggcaaaattaccgatgatgaaaatgttcgctatcgtgtcgtagcttcattaaaccatacggacggtgaatggaaagataccgataataaaacggtttatattgcgccgagtcttgaatgggatatttcagatcgtacccgtttaagtattttaactagttatcaaaaggatagcggcacaccaagttctaatttctatccgcaagaagggacgttacgtgcattgccggacggttcatactatagccgaaacgtaaactttggtgatccggtaaataatcatgaagagaataaacaatacagtatcggttatgaattaagtcacgatttcggcaatggcttgcgcgcaaactcaagctaccgttacagccatgtagaaaataatcaccgaggcggctacatttatccgtctgcgtatgcggcggattggtcggcagttgcgccaagtgctaacggctataatgtatcgggtcaaaccgtatttaatcaaggtaaagcgatttcacattcaaccgataaccgtattagctgggatttcaaaaatgattggttaaaaaatacggtggtagtcggtacggattatcgtcataacaaagtggacgctcaatatagcctatatgcgttaggttatacggcaaatttacgtgattatcgtgcaacttggaaccaaactgggacaccggttggcacggatacgcatattaaatctcgtcagctaggtttctatttacaaaatcaggctcgtattgcggataaatatctcttaggttttggtattcgtcatgatcgtgcccgtcaaaacgaatatacctctgatcaaacggttaaaaacaatcatacttcatattccggctcattaatgtatgaaggcgcatacggtttaaatccatactttagttatagcgaatcgttccgcttaccagtcggtttaagcggtaatcagtcgctgtatgatccgaatatcactcgccaatatgaacttggggtgaaatatctaccaacttggttagacggcgtgattacacttgccggattccgtgcgaaagataccggtgcattagtgaataatggcaccggtgcaacgatcagtagtgccgatccaatttatcgcaagggctttgagctgcaaacggatgtgaacttaactgagaattggaatgcaaccttagcttacacttatacgaaagcggaaagtgaagcggcaaccggtgagaaaactcgtcagtcattgattccgactaatactttagcggctcgtactgcttatagcttcactgatagcgtattaaacggcttaacggtgggagcgggtgtacgttatttagggcatagcgtcacttcaaaaggctcgctctattcacacgttcgtttgccaagtgcaacggtggtggatttaatggctcgttataatatcaatcaaaactggttggcacagcttaatgtggataacgtaggcaaccgtaagtatgtcgctgcttgtgactactactgctactatggtgcggaacgtaaagtcaatgccacagtaagttacaaattctaatttgcaaaacctattaaaagagctgataatttcagctcttttttatcataatcataaggaaacaatatgcaatttaaagactatttagcaaccttctcagcggttgagcatcttgccggtttaaatgtgcgtaacgcacaaggggaagtgattcatcatattccggctgtggaaggcaaattgggttcacttaaactttacaacgccttagcggaaaaatttgacggaaaattaaccgcttcagcagcacagcaaggcattgagtggtttgccgaacacgtggaagacgcaaaacaaaatgaaggtaaacacccgaatatcgacttattatttaaagttattaacgaagatttagcattaacgctagagcctgtcgctaagtaattagtatgaagtcattctcaaacaacaagcggtcagaatttgcaaaaaatttgcagaatccgaccgcttgcttttaggaaaagcgagaattatttggttaataaatctaacgcttcttggtatttcgccacggttttttcaattacgtccgccggtactttcggtgccggagcttgtttgttccagccgctgttttccaaccagtcacgcacaaattgtgtgtcgaatgacggcgggtttgtgccttcttgataggtatctaccgaccaaaaacggcttgagtcaggggttagcacttcatccattaacgtaagcgtaccattctcatctaaaccgaattcaaatttggtatcgcaaataataatgcctttagtgagcgcgtattccgccgcttcggtataaagtgcaattgccgcatcacgcacttttgccgctaattccgcaccgatttggcgttcgcattcttcgtatgaaatattgatatcgtgattgcccacttccgctttgcttgacggcgtgaaaatcggttgtggcaatttgcttgcctctactaaaccttccggtaattttaagccgcaaatcgtaccggtttgcttgtaatcttttaagcccgaaccggttaaataacctcgcacgatagattcgatttttaccggtgttaaacgtttgcaaaccacggcacggtgttgaatcgcttccgcttcttctttcggtaaaacatcaaaaacggaatcgccggtgaaatgattcggcataatatgggctaatttttggaaccagaaattagaaatttgggtgaggatctcgcctttacgtggaatcggatcatctaatatcacatcaaatgcggataaacggtcggtggcgaccatcaacatacgtttatcatcaatttcataaa

SEQ ID NO: 2 - protein, 672 aa, Artificial Sequence
  A. pleuropneumoniae FhuA sequence
QETAVLDEVSVVSSGSMYKMGEVPVHQAKSAVAISREDLDKQDVKKADEIGRYQAGFANQVFGNDTNTNWFRVRGEEVSQAVDGLPTFSYGFFTPYVKTFGLEAVEVTKGADSMTFGAANSGGLINYVTKRAHRDQIGRGEFKTNFGSNNLYGFGADYTGKITDDENVRYRVVASLNHTDGEWKDTDNKTVYIAPSLEWDISDRTRLSILTSYQKDSGTPSSNFYPQEGTLRALPDGSYYSRNVNFGDPVNNHEENKQYSIGYELSHDFGNGLRANSSYRYSHVENNHRGGYIYPSAYAADWSAVAPSANGYNVSGQTVFNQGKAISHSTDNRISWDFKNDWLKNTVVVGTDYRHNKVDAQYSLYALGYTANLRDYRATWNQTGTPVGTDTHIKSRQLGFYLQNQARIADKYLLGFGIRHDRARQNEYTSDQTVKNNHTSYSGSLMYEGAYGLNPYFSYSESFRLPVGLSGNQSLYDPNITRQYELGVKYLPTWLDGVITLAGFRAKDTGALVNNGTGATISSADPIYRKGFELQTDVNLTENWNATLAYTYTKAESEAATGEKTRQSLIPTNTLAARTAYSFTDSVLNGLTVGAGVRYLGHSVTSKGSLYSHVRLPSATVVDLMARYNINQNWLAQLNVDNVGNRKYVAACDYYCYYGAERKVNATVSYKF

SEQ ID NO: 3 - protein, 725 aa, Artificial Sequence
  E. coli FhuA
AVEPKEDTITVTAAPAPQESAWGPAATIAARQSATGTKTDTPIQKVPQSISVVTAEEMALHQPKSVKEALSYTPGVSVGTRGASNTYDHLIIRGFAAEGQSQNNYLNGLKLQGNFYNDAVIDPYMLERAEIMRGPVSVLYGKSSPGGLLNMVSKRPTTEPLKEVQFKAGTDSLFQTGFDFSDSLDDDGVYSYRLTGLARSANAQQKGSEEQRYAIAPAFTWRPDDKTNFTFLSYFQNEPETGYYGWLPKEGTVEPLPNGKRLPTDFNEGAKNNTYSRNEKMVGYSFDHEFNDTFTVRQNLRFAENKTSQNSVYGYGVCSDPANAYSKQCAALAPADKGHYLARKYVVDDEKLQNFSVDTQLQSKFATGDIDHTLLTGVDFMRMRNDINAWFGYDDSVPLLNLYNPSSHHHHHHGSSVNTDFDFNAKDPANSGPYRILNKQKQTGVYVQDQAQWDKVLVTLGGRYDWADQESLNRVAGTTDKRDDKQFTWRGGVNYLFDNGVTPYFSYSESFEPSSQVGKDGNIFAPSKGKQYEVGVKYVPEDRPIVVTGAVYNLTKTNNLMADPEGSFFSVEGGEIRARGVEIEAKAALSASVNVVGSYTYTDAEYTTDTTYKGNTPAQVPKHMASLWADYTFFDGPLSGLTLGTGGRYTGSSYGDPANSFKVGSYTVVDALVRYDLARVGMAGSNVALHVNNLFDREYVASCFNTYGCFWGAERQVVATATFRF

SEQ ID NO: 4 - DNA, 2739 bp, Artificial Sequence
  2886-bp coding sequence of the hgbA of Actinobacillus pleuropneumoniae serotype 1
atgattgtgattaagcttcgttttaatctatctattgcagcactttgtgttttgcaatacaacagtgcattggcacaagagcaaatgcaactcgacaccgtgattgtaaaggacggtattcagcaacaaaaaatttctaatctgattgtaaaggacggtattcagcaacaaaaaatttctgaaatcaaaaaatctgcaaaacaactggcaaaacagcaagtacaagactcacgagatctcgtacgttatgaaaccggtgtgacggtagttgaaaccggacgtatggggagtagcggttatgcggtgcgaggggtagatgagaaccgtgttgcaattacagtagatggtttacatcaagcggaaacactttcatctcagggttttaaagaattatttgaagggtatggcaattttaataatacccgtaactcggtagaaattgaaacactgaaacaagcgacgattcgtaaaggtgcggattccgttcgtgtcggtagcggtgctttaggcggcgcagtaatgtttgaaaccaaagatgctcgtgattttttaacggaaaaagattaccactttggttataaaaccggttattcaagtgcagataatcaaaaagccagctcactgacctttgccggtcgttataaaaattttgatattttagcgttaaaaacttggcgtgacggtcatgaattggaaaactatgattacaagacggctgacggtagtgtacaagggaaagaacgagaaaaagccgacccttattcaattaagaaagacagtactttaattaaattcggttaagaaaaatagaggccatgacttttcttacaacttaaaacagacaacttatattaacgttgacgaatatgagttacgtcatacgaatgataaaagtaaacgccagaattatgcttttgtgtatgagaattttagtgcgaatccattatgggatacattaaagcttacctattcaacacaaaaaattaaaaatcgtgctaggcggtttacagctaaaagatggtaaagtggttgatagagacggtaatcaaccgaacttaggcgtggatgaattaggtttaaccacaattaccaattcaaaaggcacttatactgagggagtaaatctcgttagagcttattggtttgattgtagtgtttttgattgtaataaatctgtcacagcatattataaagattattcaaataatataacctcggaagaggttgcacttacaaaaacgtatacggacgaaaaaggacgtaagtttgcaactttagatccaaagagtaaatttaaaagcatcctattaccaggaagtaaaggctatacggaaaatatttatactgaacgagatttagatacggatacaaagcaactgaatcttgatttaactaaatccttctctttactcggtcaagaacattcattggaatatggtggtagctatagccgtacagagaaaaaaatggttaaccgtggcggtttcggtgcaacgagtaatactcaatggtggacaaagcgtttccttggtatgcggaataacttctttaaaggaacggaagaagtcattacttgtaaaaatgcgaccggtagcgatcaatggaatggcttaatttgcccgaatgaagatacgttttcgttcttaattccggttgaagcgaaaaatggttcattatattttgcggataatttgaaagcgaatgactatttaagttttgatatcggctaccgctatgatgatattagctataagcctaaatatgttgcgggacaaacggcgaagattccggatgatatgccgccaaaatgcggaagaaaatattaaatatttatcacgtaatgttaaatatcaaaaacattcttattcattagcggcaactgcggatccgtttagcttcttgcgcgtacaagttaaatacgcaaatggatttagagcaccgacttctgatgagctttattttacctttaagcacccagattttaccgtattgcctaatgtggatcttaaatctgagcgtgcagagacgaaagaagccgcattaactttacatggtgatttaggctttattacaaccagttactttaaaacggattatgataattttattgatttgaagtatcttggtcctaaaaatctaatgaatgctttcggcggcagtgcaacggcaagaccttatcaaatttatcaaaatgtaaaccgtcaagaggctagagttaacggcttagaaattagttctaaattgaatttcgggcaattatggcaagctttagaagggttcaacgcaagttataaatacacttatcaaaaaggtaagatggaggggaatttaccgcttaatgcgattcagccgagaacttctgtgtttggtttaggttatgcacatccggaagataaattcggtgtagatgtatatgttacccgtgtaagtgcgaagaaagcggaagatacttacaatatgtatcacagcgaagaaaaagcaaaagacagctacttaaaatggcgtagcgatgcttatacgttgttagatatgattgcttatgttaaaccgtggaaaaatgtcaccttgcaagcaggtgtttataatttacagaatcgaaaatatctgacttgggaatcagttcgttcaattcgtccgtttggcacgagtaacttgattaaccaagcaacaggtaaaggcttaaatcgtttcactgctccgggaagaaactttaagttaaccgcagaggttactttctaa

SEQ ID NO: 5 - DNA, 21 bp, Artificial Sequence
  foA primer
cacccgttaaacggcgagcac

SEQ ID NO: 6 - DNA, 16 bp, Artificial Sequence
  F7 primer
gcaaccgtccaatcca

SEQ ID NO: 7 - DNA, 18 bp, Artificial Sequence
  R7 primer
catcctgaaaccaaacga

SEQ ID NO: 8 - DNA, 31 bp, Artificial Sequence
  FA1 primer
ctcgagctcgaggcagcagcagccgtcaggc

SEQ ID NO: 9 - DNA, 19 bp, Artificial Sequence
  5T3W1 primer
cattgaaaacctcgagtcg

SEQ ID NO: 10 - DNA, 18 bp, Artificial Sequence
  5T7W1 primer
gatacctatcaagaaggc

SEQ ID NO: 11 - DNA, 18 bp, Artificial Sequence
  5T3W2 primer
tggcgcaacaaagcaaat

SEQ ID NO: 12 - DNA, 18 bp, Artificial Sequence
  5T7W2 primer
gttgcctacgttatccac

SEQ ID NO: 13 - DNA, 18 bp, Artificial Sequence
  5T3W3 primer
accgttgttcagccttat

SEQ ID NO: 14 - DNA, 18 bp, Artificial Sequence
  5T7W3 primer
gagcgtccactttgtatg

SEQ ID NO: 15 - DNA, 19 bp, Artificial Sequence
  5T3W4 primer
agttgttgagtttggcgat

SEQ ID NO: 16 - DNA, 18 bp, Artificial Sequence
  5T7W4 primer
tcattgagtctgcgcctt

SEQ ID NO: 17 - DNA, 18 bp, Artificial Sequence
  5T3W5 primer
tggtagcaatagcggtcg

SEQ ID NO: 18 - DNA, 18 bp, Artificial Sequence
  AFor primer
gatgaggtgtcggtggtt

SEQ ID NO: 19 - DNA, 18 bp, Artificial Sequence
  ARev primer
tgtggcattgactttacg

SEQ ID NO: 20 - DNA, 19 bp, Artificial Sequence
  BFor primer
cttattcagcggattagcc

SEQ ID NO: 21 - DNA, 20 bp, Artificial Sequence
  BRev primer
caactaatgtggcgactaag

SEQ ID NO: 22 - DNA, 19 bp, Artificial Sequence
  CFor primer
gcaattcgagcagggtaag

SEQ ID NO: 23 - DNA, 19 bp, Artificial Sequence
  CRev primer
ccggtcgtttggtttcagg

SEQ ID NO: 24 - DNA, 18 bp, Artificial Sequence
  DFor primer
gtgagcttccgtatttct

SEQ ID NO: 25 - DNA, 18 bp, Artificial Sequence
  DRev primer
cgcctccgtgcaataatc

SEQ ID NO: 26 - DNA, 31 bp, Artificial Sequence
  SaII/HgbA primer
gaggtcgacaacagtgcattggctcaagagc

SEQ ID NO: 27 - DNA, 31 bp, Artificial Sequence
  XhoI/HgbA primer
gcgctcgaggaaagtaacctctgcggttaac

SEQ ID NO: 28 - protein, 11 aa, Artificial Sequence
  peptide from hgbA
QEQMQLDTVKV

SEQ ID NO: 29 - protein, 8 aa, Artificial Sequence
  peptide from hgbA
NSVEITLK

SEQ ID NO: 30 - protein, 12 aa, Artificial Sequence
  peptide from hgbA
APTSDELYFTFK

SEQ ID NO: 31 - protein, 9 aa, Artificial Sequence
  peptide from hbgA
FGVDVYVTR

SEQ ID NO: 32 - protein, 8 aa, Artificial Sequence
  ATP/GTP binding site motif A
GHNGSGKS

SEQ ID NO: 33 - protein, 15 aa, Artificial Sequence
  ABC transporter family signature amino acids 138-152
LSGGERSRIWLAMLL

SEQ ID NO: 34 - protein, 15 aa, Artificial Sequence
  ABC transporter family signature amino acids 489-503
IASGDPRANQLITWT

SEQ ID NO: 35 - protein, 13 aa, Artificial Sequence
  target TbpA
EQAVQLNDVYVTG

SEQ ID NO: 36 - protein, 13 aa, Artificial Sequence
  target FhuA
QETAVLDEVSVVS

SEQ ID NO: 37 - protein, 13 aa, Artificial Sequence
  target HgbA
QEQMQLDTVIVKD

SEQ ID NO: 38 - DNA, 6 bp, Artificial Sequence
  putative box at -10 in fhu gene
taatta

SEQ ID NO: 39 - DNA, 4 bp, Artificial Sequence
  Shine-Dalgarno sequence
ggag

SEQ ID NO: 40 - DNA, 5 bp, Artificial Sequence
  consensus sequence for the -35 region in fhu gene
tttaa

SEQ ID NO: 41 - DNA, 19 bp, Artificial Sequence
  consensus sequence for the Fur box
gataatgataatcattatc